(19) United States
(12) Patent Application Publication
(10) Pub. No.: US 2023/0074545 A1
(43) Pub. Date: Mar. 9, 2023

(54) TREATMENT OF CANCER WITH CDK12/13 INHIBITORS

(71) Applicant: Kinnate Biopharma Inc., San Diego, CA (US)

(72) Inventors: Eric A. MURPHY, San Marcos, CA (US); John TYHONAS, San Diego, CA (US); Noelito TIMPLE, San Diego, CA (US); Toufike KANOUNI, Rancho Santa Fe, CA (US); Lee D. ARNOLD, Rancho Santa Fe, CA (US); Elisabeth GARDINER, San Diego, CA (US); Eric MARTIN, Del Mar, CA (US)

(21) Appl. No.: 17/789,484

(22) PCT Filed: Dec. 23, 2020
(86) PCT No.: PCT/US2020/066967

§ 371 Date: Jun. 27, 2022

Related U.S. Application Data
(60) Provisional application No. 62/956,114, filed on Dec. 31, 2019.

(51) Int. Cl. A61K 31/506; A61P 35/04; A61P 35/00; C12Q 1/6886

(52) U.S. Cl. CPC A61K 31/506; A61P 35/04; A61P 35/00; C12Q 1/6886; C12Q 1/6851

(57) ABSTRACT

Provided herein are compositions and methods for the treatment of a triple-negative breast cancer, ovarian cancer and castration-resistant prostate cancer. Said compositions comprise CDK12/13 inhibitors.

### CROSS-REFERENCE TO RELATED APPLICATIONS

This application claims the benefit of U.S. Provisional Patent Application No. 62/956,114 filed on Dec. 31, 2019, which is herein incorporated by reference in its entirety.

### BRIEF SUMMARY OF THE INVENTION

Provided herein are compositions and methods for the treatment of cancer. The types of cancer suitable for the methods disclosed herein include, but are not limited to, triple-negative breast cancer, ovarian cancer and castration-resistant prostate cancer. The compositions useful for the methods of treating cancer disclosed herein comprise heterocyclic CDK12/13 inhibitors described herein.

One embodiment provides a method of treating a triple-negative breast cancer in an individual in need thereof, comprising administering to the individual a compound of Formula (I), or pharmaceutically acceptable salt or solvate thereof, wherein the compound of Formula (I) has the structure:

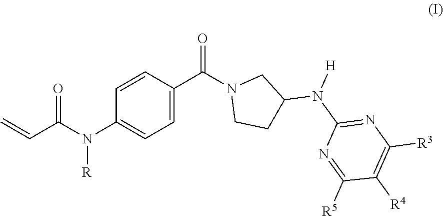

wherein,

R is hydrogen or C1-C3 alkyl;

R³ is selected from hydrogen, halogen, —CN, and optionally substituted C1-C3 alkyl;

R⁴ is selected from selected from halogen, —CN, optionally substituted alkyl, optionally substituted fluoroalkyl, optionally substituted alkenyl, optionally substituted alkynyl; and

R⁵ is hydrogen or optionally substituted alkoxy.

One embodiment provides a method of treating ovarian cancer in an individual in need thereof, comprising administering to the individual a compound of Formula (I), or pharmaceutically acceptable salt or solvate thereof, wherein the compound of Formula (I) has the structure:

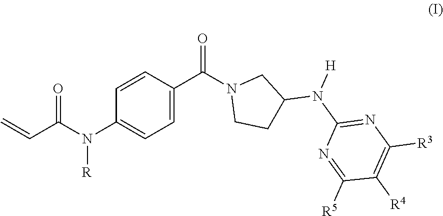

wherein,

R is hydrogen or C1-C3 alkyl;

R³ is selected from hydrogen, halogen, —CN, and optionally substituted C1-C3 alkyl;

R⁴ is selected from selected from halogen, —CN, optionally substituted alkyl, optionally substituted fluoroalkyl, optionally substituted alkenyl, optionally substituted alkynyl; and

R⁵ is hydrogen or optionally substituted alkoxy.

One embodiment provides a method of treating castration-resistant prostate cancer in an individual in need thereof, comprising administering to the individual a compound of Formula (I), or pharmaceutically acceptable salt or solvate thereof, wherein the compound of Formula (I) has the structure:

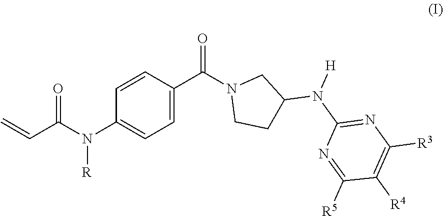

wherein,

R is hydrogen or C1-C3 alkyl;

R³ is selected from hydrogen, halogen, —CN, and optionally substituted C1-C3 alkyl;

R⁴ is selected from selected from halogen, —CN, optionally substituted alkyl, optionally substituted fluoroalkyl, optionally substituted alkenyl, optionally substituted alkynyl; and

R⁵ is hydrogen or optionally substituted alkoxy.

### BRIEF DESCRIPTION OF THE DRAWINGS

FIG. 1 displays the tumor volume over time in a mouse xenograft model of triple negative breast cancer treated with compound 4.

FIG. 2 displays the tumor volume over time in a mouse xenograft model of ovarian cancer treated with compound 4.

FIGS. 3A and 3B depict dose-response curves for THZ531 and compound 1 during in vitro analysis of inhibition of RNA II phosphorylation.

FIG. 4 depicts changes in levels of RNA II phosphorylation in a murine triple-negative breast cancer xenograft model treated with compound 4 and compound 5.

FIG. 5 depicts dose-response curves for compound 1 and compound 4 in human and cynomolgus monkey peripheral blood mononucleated cells (PBMCs).

FIG. 6 depicts changes in levels of BRCA1 expression in an analysis of compound 1 in a triple-negative breast cancer cell line.

FIG. 7 depicts changes in levels of BRCA1 expression in a murine triple-negative breast cancer xenograft model treated with compound 4.

### INCORPORATION BY REFERENCE

All publications, patents, and patent applications mentioned in this specification are herein incorporated by reference for the specific purposes identified herein.

### DETAILED DESCRIPTION OF THE INVENTION

### Certain Terminology

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and sub-combinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

“Cyano” refers to the —CN radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)OR^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O— alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each IV is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —SR′, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

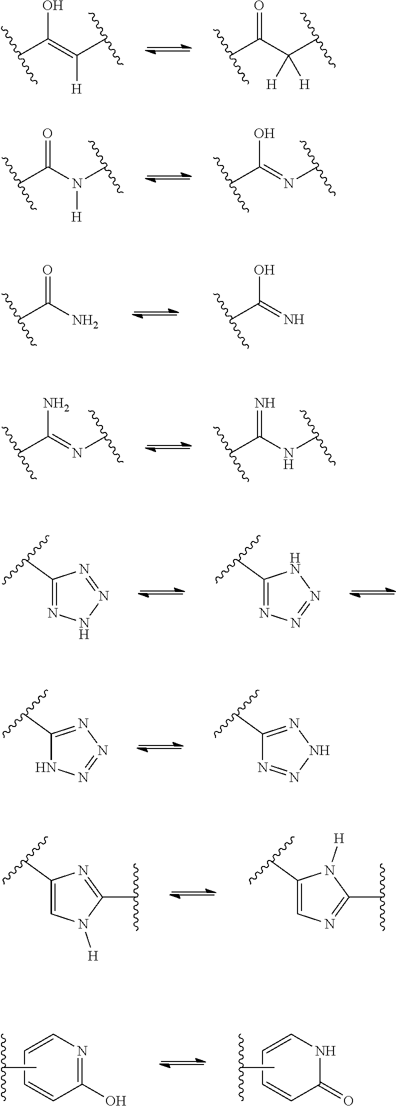

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. In some embodiments, isotopic substitution with ¹⁸F is contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

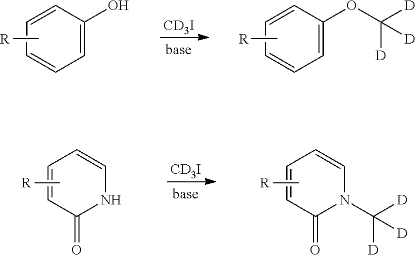

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

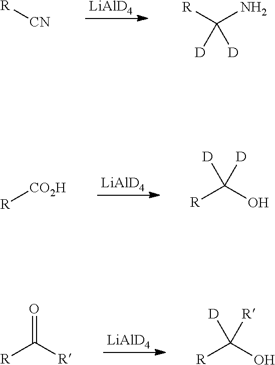

Deuterium gas and palladium catalyst are employed to reduce unsaturated carbon-carbon linkages and to perform a reductive substitution of aryl carbon-halogen bonds as illustrated, by way of example only, in the reaction schemes below.

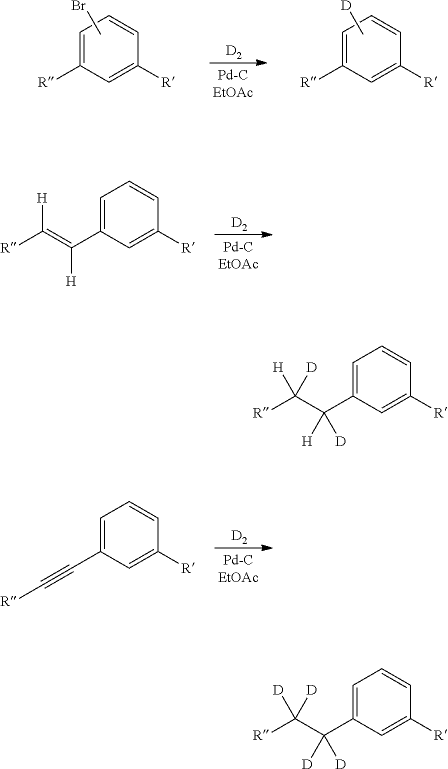

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In one embodiment, the level of deuterium incorporation is determined by synthetic methods in which a deuterated synthetic building block is used as a starting material.

“Pharmaceutically acceptable salt” includes both acid and base addition salts. A pharmaceutically acceptable salt of any one of the inhibitor of cyclin-dependent kinases (CDKs) compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. The compounds provided herein optionally exist in either unsolvated as well as solvated forms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made. The term “treating”, as used herein, unless otherwise indicated, means reversing, alleviating, inhibiting the progress of, or preventing the disorder or condition to which such term applies, or one or more symptoms of such disorder or condition. In some embodiments, the term “treating” includes slowing or delaying the progression of the disease or disorder to which the term is applied. Additionally, in some embodiments, the term “treating” is applied to one or more of the complications resulting from the disease or disorder to which the term is applied. The term “treatment”, as used herein, unless otherwise indicated, refers to the act of treating as “treating” is defined immediately above.

The term “tumor,” or “cancer” as used herein, and unless otherwise specified, refers to a neoplastic cell growth, and includes pre-cancerous and cancerous cells and tissues. Tumors usually present as a lesion or lump. As used herein, “treating” a tumor means that one or more symptoms of the disease, such as the tumor itself, vascularization of the tumor, or other parameters by which the disease is characterized, are reduced, ameliorated, inhibited, placed in a state of remission, or maintained in a state of remission. “Treating” a tumor also means that one or more hallmarks of the tumor may be eliminated, reduced or prevented by the treatment. Non-limiting examples of such hallmarks include uncontrolled degradation of the basement membrane and proximal extracellular matrix, migration, division, and organization of the endothelial cells into new functioning capillaries, and the persistence of such functioning capillaries.

The phrase “therapeutically effective amount”, as used herein, refers to that amount of drug or pharmaceutical agent that will elicit the biological or medical response of a tissue, system, animal, or human that is being sought by a researcher, veterinarian, medical doctor or other.

Other aspects, advantages, and features of the invention will become apparent from the detailed description below.

### CDK12/CDK13

The members of the cyclin-dependent kinase (CDK) family play critical regulatory roles in proliferation. In the nucleus, CDK12 and/or CDK13 can help to form the kinase core of the RNA polymerase (RNAP) II general transcription factor complex and can phosphorylate Serine 2 of the C-terminal domain (CTD) of RNAP II, which is a requisite step in gene transcriptional initiation. Together, the two functions of CDK12/13, i.e., CAK and CTD phosphorylation, support critical facets of cellular proliferation, cell cycling, and transcription. Disruption of RNAP II CTD phosphorylation has been shown to preferentially affect proteins with short half-lives, including those of the anti-apoptotic BCL-2 family. Cancer cells have demonstrated ability to circumvent pro-cell death signaling through upregulation of BCL-2 family members. Ergo, inhibition of human CDK12 and/or CDK13 kinase activity is likely to result in anti-proliferative activity in cancerous cells.

In some cases, the cancer or proliferative disease to be treated or prevented will typically be associated with aberrant activity of CDK12 and/or CDK13. Aberrant activity of CDK12 and/or CDK13 may be an elevated and/or an inappropriate (e.g., abnormal) activity of CDK12 and/or CDK13. In certain embodiments, CDK12 and/or CDK13 is not overexpressed, and the activity of CDK12 and/or CDK13 is elevated and/or inappropriate. In certain other embodiments, CDK12 and/or CDK13 is overexpressed, and the activity of CDK12 and/or CDK13 is elevated and/or inappropriate.

A proliferative disease may also be associated with inhibition of apoptosis of a cell in a biological sample or subject. All types of biological samples described herein or known in the art are contemplated as being within the scope of the invention. Inhibition of the activity of CDK12 and/or CDK13 is expected to cause cytotoxicity via induction of apoptosis.

### Heterocyclic CDK12/13 Inhibitors

In some embodiments, the heterocyclic CDK12/13 inhibitors described herein are described by a compound, or pharmaceutically acceptable salts or solvates thereof, having the structure of Formula (I):

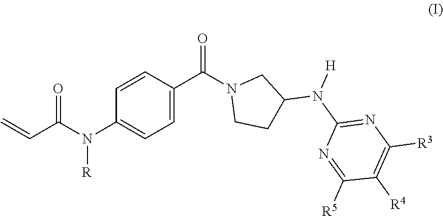

wherein,
R is hydrogen or C1-C3 alkyl;
R³ is selected from hydrogen, halogen, —CN, and optionally substituted C1-C3 alkyl;
R⁴ is selected from selected from halogen, —CN, optionally substituted alkyl, optionally substituted fluoroalkyl, optionally substituted alkenyl, optionally substituted alkynyl; and
R⁵ is hydrogen or optionally substituted alkoxy.

In some embodiments, the compound, or pharmaceutically acceptable salts or solvates thereof, of Formula (I) have R as hydrogen. In some embodiments, the compound, or pharmaceutically acceptable salt thereof, of Formula (I) have R as C1-C3 alkyl.

In some embodiments, the compound, or pharmaceutically acceptable salts or solvates thereof, of Formula (I) have R³ as hydrogen.

In some embodiments, the compound, or pharmaceutically acceptable salts or solvates thereof, of Formula (I) have R⁴ as —CN, optionally substituted alkyl, optionally substituted fluoroalkyl, optionally substituted alkenyl, optionally substituted alkynyl. In some embodiments, the compound, or pharmaceutically acceptable salts or solvates thereof, of Formula (I) have R⁴ as —CN. In some embodiments, the compound, or pharmaceutically acceptable salts or solvates thereof, of Formula (I) have R⁴ as optionally substituted alkynyl. In some embodiments, the compound, or pharmaceutically acceptable salts or solvates thereof, of Formula (I) have R⁴ as halogen.

In some embodiments, the compound, or pharmaceutically acceptable salts or solvates thereof, of Formula (I) have R⁵ as hydrogen.

In particular embodiments, the heterocyclic CDK12/13 inhibitors described herein, or pharmaceutically acceptable salts or solvates, thereof have been previously disclosed in PCT patent application PCT/US2019/039959 and related patent applications, which are incorporated by reference in their entirety. Throughout this disclosure reference is made to particular heterocyclic CDK12/13 inhibitors, or pharmaceutically acceptable salts or solvates thereof. The structures of said inhibitors are provided below in Table 1.

TABLE 1

| Compound | Structure | Name |
|---|---|---|
| 1 | 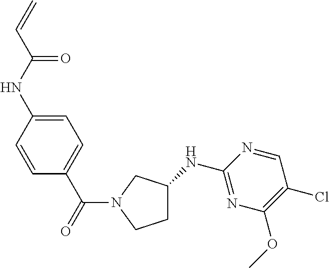 [SMILES: C=CC(=O)Nc1ccc(C(=O)N2CC[C@@H](Nc3ncc(Cl)c(OC)n3)C2)cc1] | (R)-N-(4-(3-((5-chloro-4- methoxypyrimidin-2- yl)amino)pyrrolidine-1- carbonyl)phenyl)acrylamide |
| 2 | 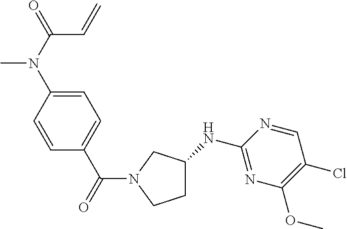 [SMILES: C=CC(=O)N(C)c1ccc(C(=O)N2CC[C@@H](Nc3ncc(Cl)c(OC)n3)C2)cc1] | (R)-N-(4-(3-((5-chloro-4- methoxypyrimidin-2- yl)amino)pyrrolidine-1- carbonyl)phenyl)-N- methylacrylamide |
| 3 | 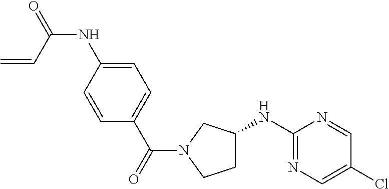 [SMILES: C=CC(=O)Nc1ccc(C(=O)N2CC[C@@H](Nc3ncc(Cl)cn3)C2)cc1] | (R)-N-(4-(3-((5- chloropyrimidin-2- yl)amino)pyrrolidine-1- carbonyl)phenyl)acrylamide |
| 4 | 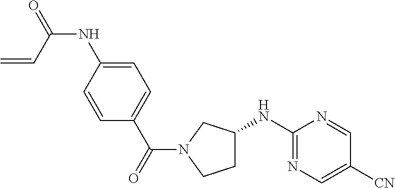 [SMILES: C=CC(=O)Nc1ccc(C(=O)N2CC[C@@H](Nc3ncc(C#N)cn3)C2)cc1] | (R)-N-(4-(3-((5- cyanopyrimidin-2- yl)amino)pyrrolidine-1- carbonyl)phenyl)acrylamide |
| 5 | 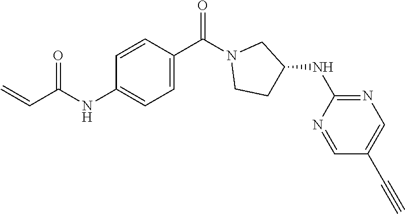 [SMILES: C#Cc1cnc(N[C@@H]2CCN(C(=O)c3ccc(NC(=O)C=C)cc3)C2)nc1] | (R)-N-(4-(3-((5- ethynylpyrimidin-2- yl)amino)pyrrolidine-1- carbonyl)phenyl)acrylamide |

In some embodiments, the compound, or pharmaceutically acceptable salt or solvate thereof, of Formula (I) is (R)—N-(4-(3-((5-chloro-4-methoxypyrimidin-2-yl)amino)pyrrolidine carbonyl)phenyl)acrylamide (compound 1), or pharmaceutically acceptable salt or solvate thereof.

In some embodiments, the compound, or pharmaceutically acceptable salt or solvate thereof, of Formula (I) is (R)—N-(4-(3-((5-chloro-4-methoxypyrimidin-2-yl)amino)pyrrolidine-1-carbonyl)phenyl)-N-methylacrylamide (compound 2), or pharmaceutically acceptable salt or solvate thereof.

In some embodiments, the compound, or pharmaceutically acceptable salt or solvate thereof, of Formula (I) is (R)—N-(4-(3-((5-chloropyrimidin-2-yl)amino)pyrrolidine-1-carbonyl)phenyl)acrylamide (compound 3), or pharmaceutically acceptable salt or solvate thereof.

In some embodiments, the compound, or pharmaceutically acceptable salt or solvate thereof, of Formula (I) is (R)—N-(4-(3-((5-cyanopyrimidine-2-yl)amino)pyrrolidine-1-carbonyl)phenyl)acrylamide (compound 4), or pharmaceutically acceptable salt or solvate thereof.

In some embodiments, the compound, or pharmaceutically acceptable salt or solvate thereof, of Formula (I) is (R)—N-(4-(3-((5-ethynylpyrimidine-2-yl)amino)pyrrolidine-1-carbonyl)phenyl)acrylamide (compound 5), or pharmaceutically acceptable salt or solvate thereof.

### Cancer and Methods of Treatment

In certain aspects, disclosed herein is a method of treating a cancer in an individual in need thereof, comprising administering an effective amount of a heterocyclic CDK12/13 inhibitor described herein to the individual. In certain aspects, disclosed herein is a heterocyclic CDK12/13 inhibitor for use in treating a cancer. In certain aspects, disclosed herein is a heterocyclic CDK12/13 inhibitor for use in preparation of a medicament for treating a cancer.

In certain embodiments, the cancer is a hormone dependent cancer such as breast, prostate or ovarian cancer. In certain embodiments, the cancer is a hormone resistant form of breast, prostate or ovarian cancer. In certain embodiments, the cancer is a breast cancer. In certain embodiments, the cancer is a triple-negative breast cancer (TNBC). In certain embodiments, the cancer is an ovarian cancer. In certain embodiments, the cancer is a prostate cancer. In certain embodiments, the cancer is a castration-resistant prostate cancer.

Cancer may result from mutations of tumor suppressor genes, DNA damage repair (DDR) genes, or genes associated cell proliferation and survival. Tumor suppressor and DDR genes repair damaged DNA and destroy cells with DNA damage. Some examples of these genes are BRCA1 and BRCA2, as well as anti-apoptotic proteins such as BCL-2 and XIAP. Mutations in BRCA1 and BRCA2 are highly associated with hormone dependent cancers. Overexpression of genes associated with proliferation may also result in cancers. In certain embodiments, the cancer is associated with dependence on BRCA1 or BRCA2, or DDR genes, and anti-apoptotic proteins (e.g., BCL-2 and/or XIAP). In certain embodiments, the cancer is associated with overexpression of cell proliferation genes. In certain embodiments, the cancer is associated with overexpression of MYC (a gene that codes for a transcription factor that regulates cellular proliferation, differentiation and survival).

The compounds disclosed herein, in some embodiments, may be used to treat cancer selected from the group consisting of a carcinoma, including that of the breast, liver, lung, bone, colon, kidney, bladder, including osteosarcoma, high-grade serous ovarian cancer, prostate cancer, anaplastic thyroid carcinoma (ATC), triple negative breast cancer (TNBC) and tumors with the following mutations: BRCA1/BRCA2 or DDR gene mutant cancers, ETS-fusion including prostate cancer and Ewing sarcoma, cancer with ARID1A mutations and cancers with SWI/SNF complex mutations, small cell lung cancer, non-small cell lung cancer, head and neck cancer, esophageal, stomach, pancreatic, gall bladder, cervical, skin, including squamous cell carcinoma. In addition, cancer may be selected from hematopoietic tumors of lymphoid lineage, including leukemia, acute lymphoblastic leukemia, acute lymphocytic leukemia, Hodgkins lymphoma, non-Hodgkins lymphoma, B-cell lymphoma, T-cell lymphoma, hairy cell lymphoma, myeloma, mantle cell lymphoma and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; and other tumors, including seminoma, melanoma, osteosarcoma, teratocarcinoma, keratoacanthoma, xenoderma pigmentosum, thyroid follicular cancer and Kaposi's sarcoma. In addition, disease where CDK12 may be implicated, including Myotonic dystrophy type 1 (DM1), Myotonic Dystrophy type 2, Fragile X associated tremor/ataxia syndrome, amylotrophic lateral sclerosis (ALS) and frontotemporal dementia, Huntington's disease like 2, Huntington's disease, several types of Spinocerebellar Ataxia, and Spinal and Bulbar Muscular Atrophy.

### Triple-Negative Breast Cancer

Triple-negative breast cancer (TNBC) is cancer that tests negative for estrogen receptors, progesterone receptors, and excess HER2 protein. TNBC does not respond to hormonal therapy or medicines that target HER2 protein receptors. There are fewer targeted medicines that treat TNBC, and it is considered to be more aggressive and have a poorer prognosis than other forms of breast cancer. In certain aspects, disclosed herein is a method of treating a breast cancer in an individual in need thereof, comprising administering an effective amount of a compound of Formula (I), or pharmaceutically acceptable salts or solvates thereof, to the individual. In certain aspects, disclosed herein is a method of treating a triple-negative breast cancer in an individual in need thereof, comprising administering an effective amount of a compound of Formula (I), or pharmaceutically acceptable salts or solvates thereof, to the individual. In certain aspects, disclosed herein is a compound of Formula (I), or pharmaceutically acceptable salts or solvates thereof, for use in treating a triple-negative breast cancer. In certain aspects, disclosed herein is a compound of Formula (I), or pharmaceutically acceptable salts or solvates thereof, for use in preparation of a medicament for treating a triple-negative breast cancer.

TNBC is more likely to metastasize than other forms of breast cancer. In some embodiments, the triple-negative breast cancer is a metastatic triple-negative breast cancer. In some embodiments, the triple-negative breast cancer is a non-metastatic triple-negative breast cancer.

Breast cancers can begin in many locations within the breast. Ductal carcinomas form in the lining of the breast milk duct. Paget's disease of the breast starts in the breast ducts and spreads to the nipple and the areola. Angiosarcoma starts in the cells that line blood vessels or lymph vessels. Phyllodes tumors develop in the connective tissue of the breast. Basal-like cancers, which resemble the basal cells that line the breast ducts, tend to be more aggressive and higher-grade cancers. In some embodiments, the breast cancer comprises a ductal carcinoma, Paget's disease of the breast, an angiosarcoma, a phyllodes tumor, or a basal-like cancer. Many TNBC are “basal-like” cancers. In some embodiments, the triple-negative breast cancer comprises a basal-like tumor.

People with an inherited BRCA1 or BRCA2 mutation are more likely to have TNBC. In some embodiments, the individual has a BRCA1 mutation. In some embodiments, the individual has a BRCA2 mutation.

### Ovarian Cancer

In certain aspects, disclosed herein is a method of treating an ovarian cancer in an individual in need thereof, comprising administering an effective amount of a compound of Formula (I), or pharmaceutically acceptable salts or solvates thereof, to the individual. In certain aspects, disclosed herein is a compound of Formula (I), or pharmaceutically acceptable salts or solvates thereof, for use in treating ovarian cancer. In certain aspects, disclosed herein is a compound of Formula (I), or pharmaceutically acceptable salts or solvates thereof, for use in preparation of a medicament for treating ovarian cancer. In certain embodiments, the ovarian cancer comprises a metastatic ovarian cancer. In certain embodiments, the ovarian cancer comprises a non-metastatic ovarian cancer. In certain embodiments, the ovarian cancer is a high-grade tumor. In certain embodiments, the ovarian cancer is recurrent ovarian cancer.

Ovarian cancers form from many cell types within the ovaries. Epithelial tumors develop from the cells that cover the outer surface of the ovary. Benign epithelial tumors include, without limitations serous adenomas, mucinous adenomas, and Brenner tumors. The most common form of ovarian tumor is the cancerous epithelial carcinoma. Germ cell tumors develop from the cells that produce the ova and can be benign or cancerous. Some examples of common germ cell tumors include maturing teratomas, dysgerminomas, and endodermal sinus tumors. Stromal tumors develop from the connective tissues in the ovary that produce hormones of the ovary. Ovarian sarcomas develop in the connective tissues of ovarian cells and include adenosarcoma, leiomyosarcoma, and fibrosarcoma. In certain embodiments, the cancer comprises an ovarian epithelial cancer, a germ cell tumor, a stromal tumor, or an ovarian sarcoma. In certain embodiments, the ovarian sarcoma is an adenosarcoma, leiomyosarcoma or a fibrosarcoma. In certain embodiments, the cancer comprises an epithelial carcinoma.

BRCA1 and BRCA2 mutations increase the risk of an individual developing ovarian cancer. In certain embodiments, the individual has a BRCA1 mutation. In certain embodiments, the individual has a BRCA2 mutation.

In certain embodiments, the cancer is an epithelial ovarian cancer. In certain embodiments, the cancer is a fallopian tube cancer. In certain embodiments, the cancer is a peritoneal cancer.

### Castration-Resistant Prostate Cancer

In certain aspects, disclosed herein is a method of treating a prostate cancer in an individual in need thereof, comprising administering a compound of Formula (I), or pharmaceutically acceptable salts or solvates thereof, to the individual. In certain aspects, disclosed herein is a compound of Formula (I), or pharmaceutically acceptable salts or solvates thereof, for use in treating a prostate cancer. In certain aspects, disclosed herein is a compound of Formula (I), or pharmaceutically acceptable salts or solvates thereof, for use in preparation of a medicament for treating a prostate cancer. Many early-stage prostate cancers require normal levels of testosterone to grow, but castration-resistant prostate cancers do not. This limits the treatment options available for treatment. In certain embodiments, the prostate cancer comprises a castration resistant prostate cancer. In certain embodiments, the castrate-resistant prostate cancer comprises a metastatic prostate cancer. In certain embodiments, the castrate-resistant prostate cancer comprises a non-metastatic prostate cancer.

Prostate cancers can occur in many tissues of the gland and include, without limitations, acinar adenocarcinoma, ductal adenocarcinoma, transitional cell cancer, squamous cell cancer, small cell prostate cancer, neuroendocrine cancer, and sarcoma. Most prostate cancers are adenocarcinomas. Within this subset, most adenocarcinomas are acinar adenocarcinomas, which form in the acini cells which form clusters and line fluid secreting cells. Ductal adenocarcinomas form in the cells that line the tubes and ducts of the prostate glands. Transitional cell carcinoma forms in the structures surrounding the prostate, for example in the cells lining the urethra and the bladder. Squamous cell carcinoma is a rare and aggressive form of prostate cancer that begins in the flat cells that cover the prostate gland. Small cell carcinomas are an aggressive form of prostate cancer that develops in the small round cells of the neuroendocrine system. Neuroendocrine cancers form in the nerve and gland cells of the prostate. Sarcomas are a rare form of prostate cancer and form in the soft tissue, including muscles and nerves, of the prostate. Prostate sarcomas can include leiomyosarcomas and rhabdomyosarcomas. In certain embodiments, the castrate-resistant prostate cancer comprises acinar adenocarcinoma, ductal adenocarcinoma, transitional cell cancer, squamous cell cancer, small cell prostate cancer, a neuroendocrine cancer, or a sarcoma.

BRCA1 and BRCA2 genes increase the risk of an individual developing prostate cancer. In certain embodiments, the individual has a BRCA1 mutation. In certain embodiments, the individual has a BRCA2 mutation.

One embodiment provides a method for inducing apoptosis in a cell comprising administering to the cell an effective amount of a composition comprising a compound of Formula (I), or pharmaceutically acceptable salts or solvates thereof.

One embodiment provides a method for decreasing cell proliferation in a cell comprising administering to the cell an effective amount of a composition comprising compound of Formula (I), or pharmaceutically acceptable salts or solvates thereof.

### Biomarkers of Efficacy

It may be useful to monitor the effectiveness of the treatment. The effectiveness of the treatment may be monitored, for example, by monitoring the phosphorylation states of certain proteins or expression levels of certain genes. Information about the effectiveness of the treatment may be used to, for example, determine the prognosis of an individual treated or inform a decision to continue treatment.

### CDK12 Phosphorylation Targets

CDK12 has been found to phosphorylate the C-terminal domain of RPB1, the largest subunit of RNA polymerase II. This phosphorylation plays a major role in transcription, RNA processing, and genome stability. CDK12 has also been found to phosphorylate MAPK3, SOS1, ARHGAP35, ANKS1A, JANK2, MAPK1, BCAR3, NUP214, TPR, AHNAK, DDX20, PARD2, SEPT7, ADAM17, CLASP2, XPC, POLA1, CTNNA1, and ARFIP1. Monitoring the levels of phosphorylation of CDK12 targets in the cells of the cancer or the individual may provide feedback on the efficacy of the treatment method. It is anticipated that CDK13 utilizes many of the same phosphorylation substrates as CDK12.

In some embodiments disclosed herein are methods comprising: monitoring the level of phosphorylated CDK12 phosphorylation target and the level of total CDK12 phosphorylation target in the tumor (including, but not limited to, circulating tumor cells) and in normal tissues; determining a ratio of the two values; and observing a decrease in the level of phosphorylated CDK12 phosphorylation target relative to the total level of CDK12 phosphorylation target after administration of the compound. In some embodiments disclosed herein are methods comprising: monitoring the level of phosphorylated CDK12 phosphorylation target and the level of total CDK12 phosphorylation target in the tumor; determining a ratio of the two values; and observing a decrease in the level of phosphorylated CDK12 phosphorylation target relative to the total level of CDK12 phosphorylation target after administration of the compound, wherein the decrease in the level of phosphorylated CDK12 phosphorylation target relative to the total level of RNA polymerase after administration of the compound correlates to an efficacy of the treatment. In some embodiments, the CDK12/13 phosphorylation target is selected from the group consisting of MAPK3, SOS1, ARHGAP35, ANKS1A, JANK2, MAPK1, BCAR3, NUP214, TPR, AHNAK, DDX20, PARD2, SEPT7, ADAM17, CLASP2, XPC, POLA1, CTNNA1, and ARFIP1. In some embodiments, the CDK12/13 phosphorylation target is RNA polymerase II. It is anticipated that CDK13 utilizes many of the same phosphorylation targets as CDK12.

In some embodiments disclosed herein are methods comprising: monitoring the level of phosphorylated RNA polymerase II and the level of total RNA polymerase II in the tumor; determining a ratio of the two values; and observing a decrease in the level of phosphorylated RNA polymerase II relative to the total level of RNA polymerase II after administration of the compound. In some embodiments disclosed herein are methods comprising: monitoring the level of phosphorylated RNA polymerase II and the level of total RNA polymerase II in the tumor; determining a ratio of the two values; and observing a decrease in the level of phosphorylated RNA polymerase II relative to the total level of RNA polymerase II after administration of the compound, wherein the decrease in the level of phosphorylated RNA polymerase II relative to the total level of RNA polymerase after administration of the compound correlates to an efficacy of the treatment.

### DNA Damage Response Genes

DNA damage response (DDR) genes are involved in maintaining genome fidelity by regulating cell checkpoints and DNA repair processes. The DDR detects DNA damage and triggers a complex response that decides cell fate, by promoting cell-cycle arrest and DNA repair, or cell death in cases where DNA lesions persist and are unreconcilable. The anti-cancer activity of many chemotherapy drugs relies on the induction of DNA double-strand breaks, and tumors with mutations in DDR proteins are particularly sensitive to DNA-damaging chemotherapy. Some examples of DDR genes include, but are not limited to, BRCA1, BRCA2, ATM, ATR, H2AX, RAD51, BCLXL, BCL2MCL1, MYC, B2M, PARP1, PARP2, CHEK1 and CHEK2.

CDK12 is involved in regulating DDR genes and inhibition of CDK12 can result in the downregulation of DDR genes and proteins. In addition to inducing death, monitoring the level of expression of DDR genes could provide a method of monitoring the efficacy of a CDK12 inhibitor administered to an individual. It is anticipated that a CDK13 inhibitor will behave in a similar fashion as CDK12 inhibitor with regards to DDR gene response.

In some embodiments disclosed herein are methods comprising monitoring the level of expression of a DNA damage response gene in the tumor and normal tissues; and observing a decrease in the level of expression of the DNA damage response gene after administration of the compound. In some embodiments disclosed herein are methods comprising: monitoring the level of expression of a DNA damage response gene in the tumor and normal tissues; and observing a decrease in the level of expression of the DNA damage response gene after administration of the compound; wherein the decrease in the level of expression of the DNA damage response gene after administration of the compound correlates to an efficacy of the treatment. In some embodiments, the DNA damage response gene comprises BRCA1, BRCA2, ATM, ATR, H2AX, RAD51, BCLXL, BCL2, MCL1, MYC, B2M, PARP1, PARP2, CHEK1, or CHEK2.

In certain embodiments, the level of expression of the DDR gene is monitored by quantitative, Real-Time PCR (qRT-PCR or qPCR). In certain embodiments, the level of expression of the DDR gene is monitored by a microarray analysis or ‘Next-Generation’ sequencing (NGS) technologies including, but not limited to, RNAseq. In certain embodiments, the level of DNA damage as a function of changes in DDR gene expression in tumor and normal tissue will be monitored by, but not limited to NGS technologies (e.g. whole-genome sequencing or exome-sequencing)

### Pharmaceutical Compositions

In certain embodiments, the heterocyclic CDK12/13 inhibitor described herein is administered as a pure chemical. In other embodiments, the heterocyclic CDK12/13 inhibitor described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice.

Provided herein is a pharmaceutical composition comprising at least one heterocyclic CDK12/13 inhibitor as described herein, or a stereoisomer, pharmaceutically acceptable salt, hydrate, or solvate thereof, together with one or more pharmaceutically acceptable carriers. The carrier(s) (or excipient(s)) is acceptable or suitable if the carrier is compatible with the other ingredients of the composition and not deleterious to the recipient (i.e., the subject or the patient) of the composition.

One embodiment provides a pharmaceutical composition comprising a pharmaceutically acceptable excipient and a compound of Formula (I), or a pharmaceutically acceptable salt or solvate thereof.

One embodiment provides a method of preparing a pharmaceutical composition comprising mixing a compound of Formula (I), or a pharmaceutically acceptable salt or solvate thereof, and a pharmaceutically acceptable carrier.

Provided herein is the method wherein the pharmaceutical composition is administered orally. Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, Pa. (2005)).

Provided herein is the method wherein the pharmaceutical composition is administered by injection. In some embodiments, the heterocyclic CDK12/13 inhibitor as described by Formula (I), or pharmaceutically acceptable salt or solvate thereof, is formulated for administration by injection. In some instances, the injection formulation is an aqueous formulation. In some instances, the injection formulation is a non-aqueous formulation. In some instances, the injection formulation is an oil-based formulation, such as sesame oil, or the like.

The dose of the composition comprising at least one heterocyclic CDK12/13 inhibitor as described herein differs depending upon the subject or patient's (e.g., human) condition. In some embodiments, such factors include general health status, age, and other factors. Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient. Oral doses typically range from about 1.0 mg to about 1000 mg, one to four times, or more, per day.

### EXAMPLES

These examples are provided for illustrative purposes only and not to limit the scope of the claims provided herein.

### Example 1: Determination of Cyclin Dependent Kinase Inhibitory Activity

The compounds 1-5 (see Table 1) were assayed for kinase activity at ProQinase (ProQinase GmbH, Reaction Biology; Freiburg, Germany) using their commercially available radiometric kinase assay services. Briefly, the compounds were provided as solids and were dissolved to 1×10⁻⁰³ M/100% DMSO stock solutions on the day of assay. All protein kinases were provided by ProQinase and were expressed in Sf9 insect cells or in E. coli as recombinant GST-fusion proteins or His-tagged proteins, either as full-length or enzymatically active fragments. All kinases were produced from human cDNAs and purified by either GSH-affinity chromatography or immobilized metal. The purity of the protein kinases was examined by SDS-PAGE/Coomassie staining, the identity was checked by mass spectroscopy. A radiometric protein kinase assay (³³PanQinase® Activity Assay) was used for measuring the kinase activity of the protein kinase. All kinase assays were performed in 96-well FlashPlates™ from PerkinElmer (Boston, Mass., USA) in a 50 microliter reaction volume. The reaction cocktail was pipetted in four steps in the following order: 1) 25 microliter of assay buffer (standard buffer/[gamma-³³P]-ATP); 2) 10 microliter of ATP solution (in H₂O); 3) 5 microliter of test compound (in 10% DMSO); 4) 10 microliter of enzyme/substrate mixture. The assay for the protein kinase contained 70 mM HEPES-NaOH pH 7.5, 3 mM MgCl2, 3 mM MnCl2, 3 microM Na-orthovanadate, 1.2 mM DTT, 50 μg/ml PEG20000, ATP (variable concentrations, corresponding to the apparent ATP-Km of the kinase. The reaction cocktails were incubated at 30° C. for 60 minutes. The reaction was stopped with 50 microliter of 2% (v/v) H₃PO₄, plates were aspirated and washed two times with 200 microliter 0.9% (w/v) NaCl. Incorporation of ³³Pi was determined with a microplate scintillation counter (Microbeta, Wallac).

As a parameter for assay quality, the Z′-factor (Zhang et al., J. Biomol. Screen. 2: 67-73, 1999) for the low and high controls of each assay plate (n=8) was used. ProQinase's criterion for repetition of an assay plate is a Z′-factor below 0.4. The results are shown below in Table 2.

TABLE 2
Inhibition of CDK Activity (molar
inhibition in a radiometric assay)

| Compound | CDK12 | CDK13 | CDK2 | CDK7 | CDK9 |
|---|---|---|---|---|---|
| 1 | 2.13E−08 | 2.58E−08 | 1.15E−06 | 1.10E−06 | 2.88E−07 |
| 2 | 5.44E−08 | 3.24E−07 | 1.13E−05 | 5.18E−07 | 1.32E−06 |
| 3 | 9.67E−08 | 3.79E−07 | 7.93E−06 | 2.79E−06 | 1.17E−06 |
| 4 | 1.31E−07 | 2.93E−07 | 7.55E−06 | 6.96E−06 | 3.97E−06 |
| 5 | 5.93E−08 | 1.04E−07 | 3.37E−06 | 3.77E−06 | 1.37E−06 |

### Example 2: Determination of Kinase Selectivity

Compound 4 was analyzed for selectivity against a panel of other kinases at ProQinase using their commercially available radiometric kinase assay services and the methods described in Example 1. A summary of kinase inhibition is provided in Table 3. The compound was found to have a low average percent of inhibition against non-CDK12/13 kinases.

| | Compound 4 |
|---|---|
| Kinase | % Inhibition at 1 μM |
| CDK12/CycK | 90 |
| CDK13/CycK | 87 |
| CDK7/CycH/MAT1 | 33 |
| CDK9/CycT1 | 27 |
| CDK3/CycE1 | 25 |
| MEK5 | 23 |
| CDK5/p35NCK | 23 |
| TGFBR2 | 22 |
| CDK9/CycK | 21 |
| SLK | 21 |
| CDK18/CycY | 20 |
| ROCK2 | 19 |
| CDK2/CycD1 | 18 |
| PHKG1 | 18 |
| CDK16/CycY | 17 |
| ERK7 | 17 |
| WNK1 | 17 |
| CDK1/CycA2 | 17 |
| AuroraC | 16 |
| PAK2 | 16 |
| MINK1 | 16 |
| ROCK1 | 15 |
| EEF2K | 15 |
| FER | 15 |
| EIF2AK2 | 15 |
| CDK2/CycA2 | 15 |
| PKCepsilon | 15 |
| EIF2AK3 | 14 |
| CDK6/CycD3 | 14 |
| CSF1R | 14 |
| GSG2 | 13 |
| PKA | 13 |
| PLK3 | 13 |
| BUB1B | 13 |
| MEK1 | 13 |
| CDK1/CycB1 | 13 |
| PKMzeta | 12 |
| CK1alpha1 | 12 |
| MYLK2 | 12 |
| RPS6KA2 | 12 |
| CDK17/p35NCK | 12 |
| VEGFR2 | 12 |
| PRKD2 | 12 |
| TTK | 12 |
| PAK7 | 12 |
| HIPK4 | 11 |
| CDK1/CycE1 | 11 |
| DMPK | 11 |
| MKK7 | 11 |
| MKK6 SDTD | 11 |
| AKT2 | 11 |
| MASTL | 11 |
| p38gamma | 10 |
| RPS6KA5 | 10 |
| ARK5 | 10 |
| CDK4/CycD3 | 10 |
| CDK19/CycC | 10 |
| CAMKK2 | 9 |
| RAF1 YDYD | 9 |
| S6K | 9 |
| EPHA8 | 9 |
| ERBB4 | 9 |
| ERK2 | 9 |
| MLK4 | 9 |
| DAPK2 | 9 |
| SNK | 9 |
| CDK2/CycE1 | 9 |
| CDK4/CycD1 | 9 |
| MAP3K1 | 9 |
| p38beta | 8 |
| INSR | 8 |
| VEGFR1 | 8 |
| HIPK1 | 8 |
| CAMK4 | 8 |
| MEKK2 | 8 |
| GSK3beta | 8 |
| MAPKAPK3 | 8 |
| SIK3 | 8 |
| ALK | 8 |
| TGFBR1 | 7 |
| LIMK1 | 7 |
| PKCbeta2 | 7 |
| PLK1 | 7 |
| EGFR | 7 |
| SIK1 | 7 |
| PDGFRalpha | 7 |
| STK25 | 7 |
| IKKepsilon | 7 |
| CSK | 7 |
| ACVR2A | 7 |
| STK23 | 7 |
| MUSK | 7 |
| ASK1 | 7 |
| DYRK1A | 7 |
| GRK2 | 7 |
| CDC42BPB | 7 |
| EPHA3 | 6 |
| ACK1 | 6 |
| NEK3 | 6 |
| MST4 | 6 |
| ERK5 | 6 |
| STK39 | 6 |
| COT | 6 |
| TSF1 | 6 |
| BMPR1B | 6 |
| TXK | 6 |
| TAOK3 | 6 |
| GSK3alpha | 6 |
| PDGFRbeta | 6 |
| ACVR2B | 6 |
| JNK2 | 6 |
| TYK2 | 6 |
| RPS6KA4 | 5 |
| AuroraA | 5 |
| TEC | 5 |
| CDK20/CycT1 | 5 |
| TAOK2 | 5 |
| ERBB2 | 5 |
| GRK3 | 5 |
| SGK3 | 5 |
| EPHB3 | 5 |
| CHK2 | 5 |
| CAMK2G | 5 |
| RET | 5 |
| PKMYT1 | 5 |
| p38delta | 5 |
| STK33 | 5 |
| EPHA6 | 5 |
| RIPK2 | 5 |
| INSRR | 5 |
| PYK2 | 4 |
| NLK | 4 |
| PKCbeta1 | 4 |
| MYLK3 | 4 |
| PKCnu | 4 |
| PIM3 | 4 |
| PAK6 | 4 |
| MKNK2 | 4 |
| MKK4 | 4 |
| NEK6 | 4 |
| PIM1 | 3 |
| CHK1 | 3 |
| CAMKK1 | 3 |
| ACVRL1 | 3 |
| MST3 | 3 |
| AXL | 3 |
| ULK2 | 3 |
| CDK5/p25NCK | 3 |
| PIM2 | 3 |
| JNK1 | 3 |
| TTBK2 | 3 |
| CDK8/CycC | 3 |
| MEK2 | 3 |
| MYLK | 3 |
| EPHA1 | 3 |
| PKCmu | 3 |
| p38alpha | 3 |
| DYRK1B | 3 |
| FGFR1 | 3 |
| VRK1 | 2 |
| MARK2 | 2 |
| EPHA4 | 2 |
| SYK | 2 |
| BMPR1A | 2 |
| AMPKalpha1 | 2 |
| AKT1 | 2 |
| ROS | 2 |
| TNK1 | 2 |
| PRK2 | 2 |
| FGFR4 | 2 |
| IRAK1 | 2 |
| PHKG2 | 2 |
| MAP3K7/MAP3K7IP1 | 2 |
| DNAPK | 2 |
| EPHB2 | 1 |
| AuroraB | 1 |
| DYRK2 | 1 |
| MELK | 1 |
| CDK3/CycC | 1 |
| MST1 | 1 |
| S6Kbeta | 1 |
| PAK3 | 1 |
| BTK | 1 |
| ACVR1B | 1 |
| PKCtheta | 1 |
| ITK | 1 |
| CDK6/CycD1 | 1 |
| IKKbeta | 1 |
| LTK | 1 |
| SGK2 | 1 |
| MTOR | 1 |
| BRK | 1 |
| MEKK3 | 1 |
| RPS6KA3 | 1 |
| MET | 0 |
| PBK | 0 |
| FGFR2 | 0 |
| DCAMKL2 | 0 |
| SRPK2 | 0 |
| CAMK2D | 0 |
| BRAF | 0 |
| CDK4/CycD2 | 0 |
| DAPK3 | 0 |
| SNARK | 0 |
| MAP4K5 | 0 |
| HCK | 0 |
| NEK1 | 0 |
| GRK6 | 0 |
| MST2 | 0 |
| MARK1 | 0 |
| HIPK2 | 0 |
| RIPK5 | 0 |
| MAP4K4 | 0 |
| CAMKID | 0 |
| IRAK4 | 0 |
| ABL1 | −1 |
| TRKB | −1 |
| ACVR1 | −1 |
| MAPKAPK5 | −1 |
| JAK3 | −1 |
| RON | −1 |
| PRK1 | −1 |
| MAP4K2 | −1 |
| ABL2 | −1 |
| MARK4 | −1 |
| FGR | −1 |
| NEK4 | −1 |
| TSK2 | −1 |
| RPS6KA1 | −1 |
| JAK1 | −1 |
| TLK1 | −1 |
| CK1gamma2 | −1 |
| PKN3 | −2 |
| MKNK1 | −2 |
| CLK4 | −2 |
| CLK2 | −2 |
| PKCdelta | −2 |
| BLK | −2 |
| SRMS | −2 |
| CDK20/CycH | −2 |
| NIK | −2 |
| CDC7/DBF4 | −2 |
| SGK1 | −2 |
| AKT3 | −2 |
| FES | −2 |
| FYN | −2 |
| RPS6KA6 | −3 |
| MATK | −3 |
| EPHA7 | −3 |
| EPHB1 | −3 |
| LCK | −3 |
| TYRO3 | −3 |
| PAK4 | −3 |
| RIPK4 | −3 |
| LIMK2 | −3 |
| CLK1 | −3 |
| PKCzeta | −3 |
| DYRK3 | −3 |
| ZAK | −4 |
| STK17A | −4 |
| BMX | −4 |
| PKCiota | −4 |
| BRSK1 | −4 |
| EPHB4 | −4 |
| TBK1 | −4 |
| MAP3K9 | −4 |
| KIT | −4 |
| CK2alpha1 | −4 |
| SIK2 | −4 |
| ERK1 | −4 |
| JNK3 | −4 |
| NEK9 | −5 |
| VEGFR3 | −5 |
| GRK7 | −5 |
| TTBK1 | −5 |
| YES | −5 |
| PKCalpha | −5 |
| MAP3K10 | −5 |
| NEK11 | −5 |
| PASK | −5 |
| SRC | −5 |
| WEE1 | −5 |
| EPHA2 | −5 |
| ZAP70 | −6 |
| FRK | −6 |
| PRKG2 | −6 |
| PAK1 | −6 |
| WNK3 | −6 |
| JAK2 | −6 |
| GRK5 | −6 |
| PDK1 | −7 |
| DYRK4 | −7 |
| TRKA | −7 |
| CK1delta | −7 |
| HIPK3 | −7 |
| MAP3K11 | −7 |
| PRKX | −7 |
| EPHA5 | −8 |
| HRI | −8 |
| MERTK | −8 |
| CK1gamma3 | −8 |
| FLT3 | −8 |
| WNK2 | −8 |
| IKKalpha | −8 |
| SRPK1 | −8 |
| LYN | −8 |
| NEK2 | −8 |
| CAMK2A | −9 |
| DDR2 | −9 |
| PKCgamma | −9 |
| CLK3 | −9 |
| TSSK1 | −10 |
| BRSK2 | −10 |
| TLK2 | −10 |
| TRKC | −11 |
| SAK | −11 |
| CK1gamma1 | −11 |
| PRKG1 | −12 |
| CAMK2B | −12 |
| GRK4 | −13 |
| CK1epsilon | −13 |
| CDK6/CycD2 | −14 |
| CK2alpha2 | −14 |
| PKCeta | −14 |
| LRRK2 | −14 |
| FAK | −15 |
| NEK7 | −15 |
| DAPK1 | −15 |
| FGFR3 | −16 |
| VRK2 | −16 |
| CDC42BPA | −16 |
| MARK3 | −17 |
| TIE2 | −18 |
| IGF1R | −23 |
| MAPKAPK2 | −24 |

### Example 3: The Compounds Inhibited Cell Proliferation in Ovarian Cancer Cell Lines

OVCAR-3 was used as the cell line to model for ovarian cancer in vitro and in vivo. This cell line was obtained from ATCC (Catalog #HTB-161) and is an adenocarcinoma line from the malignant ascites of a patient with progressive adenocarcinoma of the ovary and embodies a preclinical model of high-grade, serous ovarian cancer (HGSOC). OVCAR-3 has been deemed an appropriate model system in which to study drug resistance in ovarian cancer, and the presence of hormone receptors should be useful for the evaluation of hormonal therapy. OVCAR-3 is resistant to clinically relevant concentrations of adriamycin, melphalan and cisplatin.

Compounds 1-5 were tested at different concentrations (from 4 μM to 126.4 pM; 0.5 log serial dilutions) for their ability to inhibit the proliferation of OVCAR-3 cells. Cells were grown in ATCC-formulated RPMI-1640 Medium (ATCC 30-2001)+10% FBS. The cells were cultured at 37° C. in a humidified chamber in the presence of 5% CO₂. Proliferation assays were conducted over a 72 hour time period.

Cells were cultured and maintained as described above, with cells always fed on the day prior to assay. Assays were run with a 2 hour compound exposure followed by a washout and then a 72 hour proliferation time or exposed to the test compounds for 72 hours with no washout of compound. DMSO was used as a control in all wells not receiving test compound and DMSO wells were used for plate normalization. Proliferation and survival of cells was quantified based on the amount of total ATP following cell lysis as determined by using a standard assay kit used according to manufacturer's instructions. Experiments were performed in the same media used by each cell type for growth and maintenance. Cells (5×10⁴) were plated in 96 well plates for compound exposure. CellTiter-Glo® (Promega Corporation, Madison, Wis. USA) was used to assess the anti-proliferative effects of the compounds following manufacturer's directions and utilizing the reagents supplied with the CellTiter-Glo® kit.

The results of cell proliferation assays are shown in Table 4 and display the dose dependent inhibition of cell proliferation and survival in OVCAR-3 cells exposed to compounds 1-5. These compounds showed inhibition of proliferation in OVCAR-3 cells with low IC₅₀ values. Inhibition of proliferation continued in cells where the compound had been washed out after 2 hours.

TABLE 4
Inhibition of proliferation of OVCAR3 cells (IC₅₀ in M)

| | Compound ID | | | | |
|---|---|---|---|---|---|
| | 1 | 2 | 3 | 4 | 5 |
| OVCAR-3 | 4.45E−09 | 7.63E−09 | 7.45E−09 | 3.10E−08 | 1.01E−08 |
| (no washout) | | | | | |
| OVCAR-3 | 8.24E−08 | NT | 3.37E−08 | 1.79E−07 | 1.03E−07 |
| (w/washout) | | | | | |

### Example 4: The Compounds Inhibited Cell Proliferation in Breast Cancer Cell Lines

HCC70 was the cell line used as a model of triple-negative breast cancer (TNBC). This cell line was obtained from ATCC and was initiated from a primary ductal carcinoma in 1992 (Catalog #CRL-2315; mammary gland primary ductal carcinoma). The HCC-70 tumor cell line was maintained in vitro as a monolayer culture in RPMI-1640 medium supplemented with 10% heat inactivated fetal bovine serum at 37° C. in an atmosphere of 5% CO₂ in air.

Representative compounds were tested at different concentrations (from 4 μM to 126.4 pM; 0.5 log serial dilutions) for their ability to inhibit the proliferation of HCC70 cells. Cells were grown in ATCC-formulated RPMI-1640 Medium (ATCC 30-2001)+10% FBS. The cells were cultured at 37° C. in a humidified chamber in the presence of 5% CO₂. Proliferation assays were conducted over a 72 hour time period.

Cells were cultured and maintained as described above, with cells always fed on the day prior to assay. Assays were run with a 2 hour compound exposure followed by a washout and then a 72 hour proliferation time or exposed to the test compounds for 72 hours with no washout of compound. DMSO was used as a control in all wells not receiving test compound and DMSO wells were used for plate normalization. Proliferation and survival of cells was quantified based on the amount of total ATP following cell lysis as determined by using a standard assay kit used according to manufacturer's instructions. Experiments were performed in the same media used by each cell type for growth and maintenance. Cells (5×10⁴) were plated in 96 well plates for compound exposure. CellTiter-Glo® (Promega Corporation, Madison, Wis. USA) was used to assess the anti-proliferative effects of the compounds following manufacturer's directions and utilizing the reagents supplied with the CellTiter-Glo® kit.

The results of cell proliferation assays are shown in Table 5 and display the dose dependent inhibition of cell proliferation and survival in HCC70 cells exposed to compounds 1-5. These compounds showed inhibition of proliferation in HCC70 cells with low IC₅₀ values. Inhibition of proliferation continued in cells where the compound had been washed out after 2 hours.

TABLE 5
Inhibition of Proliferation of HCC70 Cells (IC₅₀ in M)

| | Compound ID | | | | |
|---|---|---|---|---|---|
| | 1 | 2 | 3 | 4 | 5 |
| HCC70 | 5.10E−09 | 1.13E−08 | 1.01E−08 | 3.23E−08 | 1.93E−08 |
| (no washout) | | | | | |
| HCC70 | 1.41E−08 | 3.80E−08 | 1.10E−07 | 1.34E−07 | 8.21E−08 |
| (w/washout) | | | | | |

### Example 5: Determination of Anti-Proliferative Activity in a Mouse Xenograph Model of Triple-Negative Breast Cancer

To test the efficacy of the compounds in vivo, studies were done in mouse xenografts. For all studies, general procedures for animal care and housing were done in accordance with the standard, Commission on Life Sciences, National Research Council, Standard operating procedures (SOPs) of Pharmaron, Inc (Beijing, China). The mice were kept in laminar flow rooms at constant temperature and humidity with 3-5 mice in each cage with bedding changed once weekly. Animals were housed in polycarbonate cage which is in the size of 300×180×150 mm³ and in an environmentally monitored, well-ventilated room maintained at a temperature of (22±3° C.) and a relative humidity of 40%-70% on a 12 hour:12 hour light:dark cycle with full spectrum lighting. Each animal was assigned an identification number; the following identification method will be applied. Cage cards were labeled with such information as study number, group, sex, dose, animal number, initiation date, study director and telephone number. Animals were identified by ear coding. Animals had free access to irradiation sterilized dry granule food during the entire study period except for time periods specified by the protocol.

Each mouse was inoculated subcutaneously on the right flank with HCC70 tumor cells (5×10⁶) in 0.1 ml of RPMI-1640 Medium and Matrigel mixture (1:1 ratio) for tumor development. The treatment started when the mean tumor size reached approximately 100-150 mm³. Mice were assigned to groups such that the mean tumor volume was the same for each treatment group. All study animals were monitored not only tumor growth but also behavior such as mobility, food and water consumption (by cage side checking only), body weight (BW), eye/hair matting and any other abnormal effect. Any mortality and/or abnormal clinical signs were recorded.

The measurement of tumor size was conducted twice weekly with a caliper and recorded. The tumor volume (mm³) is estimated using the formula: TV=a×b2/2, where “a” and “b” are long and short diameters of a tumor, respectively. The TVs were used for calculation of the tumor growth inhibition and tumor growth delay.

Protocol-required measurements and observations were recorded manually onto excel spread sheets. All statistical tests were conducted, and the level of significance was set at 5% or P<0.05. The group means, standard deviation was calculated for all measurement parameters as study designed. Two-way RM ANOVA followed by Tukeys post hoc comparisons of the means was utilized for this study.

CB-17 SCID female mice were inoculated with the HCC70 cells as the in vivo model for triple negative breast cancer. The cells were passaged and were growing in an exponential growth phase were harvested for tumor inoculation and did not exceed cell passage 5 when used. Mice bearing the HCC70 tumors were randomized into 4 groups (n=8 mice per group), 15 day post tumor implantation with an average tumor volume of 150 mm³. Animals in each group received either vehicle (3% DMSO, 0.5% acetic acid, 96.5% (20% HP-β-CD in water) or 2.5, 5.0, and 10.0 mg/kg of compound 4 biweekly (Monday, Thursday), a total of 4 doses were intravenously administered through the course of 14 day efficacy study. Immediately after every treatment dose, a saline flush of 0.1 mL was also administered. One last dose intravenously administered on Day 15 for end of study PK plasma and PD tumor collections as indicated in the protocol. Tumor volumes were measured by caliper 2 times a week and body weights of all animals were recorded throughout the study.

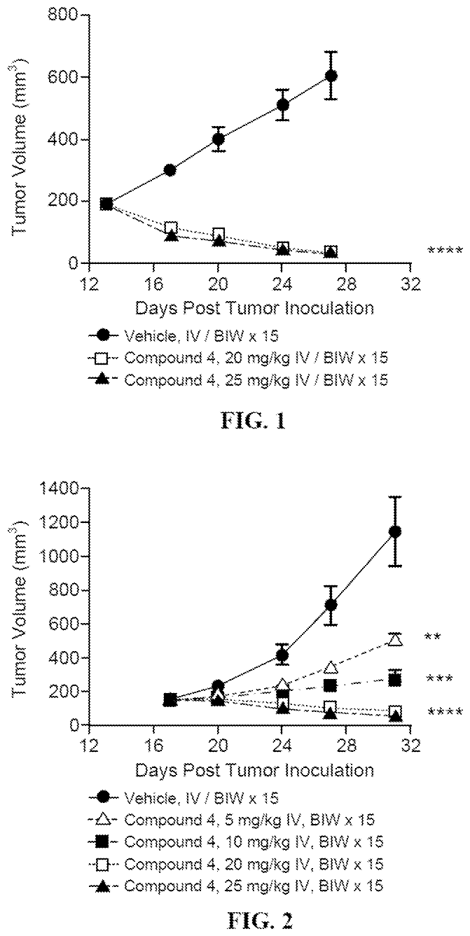

Anti-tumor activity was observed in a HCC70 xenograft model following treatment of compound 4 intravenously administered biweekly at 20 and 25 mg/kg for 14 days, as depicted in FIG. 1. At these doses, treatment with compound 4 resulted in significant anti-tumor activity with a T/C of −37% and −39% respectively, (p<0.0001, p<0.0001) compared to vehicle control. Treatment was started on day 15 post tumor inoculation. Significant differences were calculated using Two-Way RM ANOVA post hoc Tukeys post hoc comparisons of the means. Significance levels are indicated using the following scale: *=p≤0.05 **=p≤0.01 ***=p≤0.001 ****=p≤0.0001, NS=not significant.

### Example 6: Determination of Anti-Proliferative Activity in a Mouse Xenograph Model of Ovarian Breast Cancer

To test the efficacy of the compounds in vivo, studies were done in mouse xenografts. For all studies, general procedures for animal care and housing were done in accordance with the standard, Commission on Life Sciences, National Research Council, Standard operating procedures (SOPs) of Pharmaron, Inc (Beijing, China). The mice were kept in laminar flow rooms at constant temperature and humidity with 3-5 mice in each cage with bedding changed once weekly. Animals were housed in polycarbonate cage which is in the size of 300×180×150 mm³ and in an environmentally monitored, well-ventilated room maintained at a temperature of (22±3° C.) and a relative humidity of 40%-70% on a 12:12 light:dark cycle with full spectrum lighting. Each animal was assigned an identification number; the following identification method will be applied. Cage cards were labeled with such information as study number, group, sex, dose, animal number, initiation date, study director and telephone number. Animals were identified by ear coding. Animals had free access to irradiation sterilized dry granule food during the entire study period except for time periods specified by the protocol.

For the OVCAR-3 study, NOD SCID female mice bearing the OVCAR-3 tumors were randomized into 5 groups (n=9 mice per group) 17 day post tumor implantation with an average tumor volume of 150 mm³. Each group received either vehicle (3% DMSO, 97.0% (20% HP-β-CD in water) or 5.0, 10, 20 and 25.0 mg/kg of compound 4 biweekly (Monday, Thursday), a total of 4 doses were intravenously administered through the course of 14 day efficacy study. Immediately after every treatment dose, a saline flush of 0.1 mL were also administered. One last dose was intravenously administered on Day 15 for end of study PK plasma and PD tumor collections as indicated in the protocol. Tumor volumes were measured by caliper 2 times a week and body weights of all animals were recorded throughout the study.

Protocol-required measurements and observations were recorded manually onto excel spread sheets. All statistical tests were conducted, and the level of significance was set at 5% or P<0.05. The group means, standard deviation was calculated for all measurement parameters as study designed. Two-way RM ANOVA followed by Tukeys post hoc comparisons of the means was utilized for this study.

Anti-tumor activity was observed in an OVCAR-3 xenograft model following treatment of Compound 4 intravenously administered biweekly at 5.0, 10, 20, and 25 mg/kg for 14 days, as depicted in FIG. 2. At 5.0 and 10 mg/kg dose with compound 4, resulted in significant anti-tumor activity with a T/C of 36% and 13% respectively, (p<0.01, p<0.001) compared to vehicle control. At doses 20 and 25 mg/kg resulted in significant anti-tumor activity with a T/C of −7% and −9%, (p<0.0001, p<0.0001) respectively.

### Example 7: Compound 1 Inhibits Phosphorylation of RNA Polymerase II In Vitro

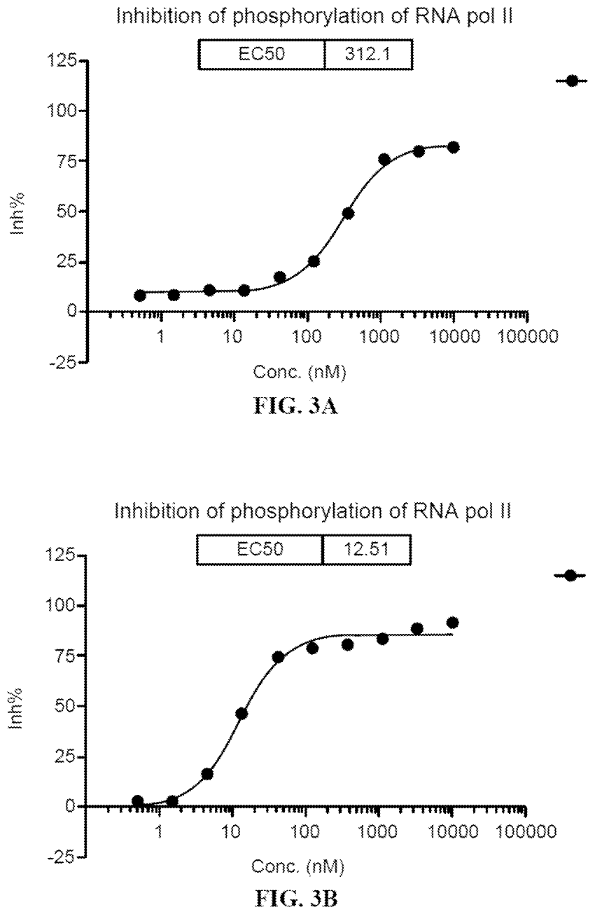

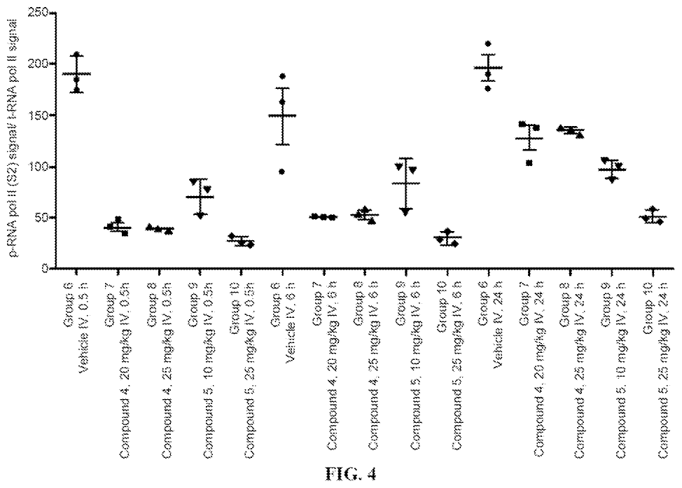

CDK12 and CDK13 phosphorylate RNA polymerase II. The ability of compound 1 to inhibit phosphorylation of RNA polymerase II was analyzed in NCI-H82 cells. 200,000 cells were plated in each well of a 96 well MSD plate. Cells were treated with compound 1, THZ531 (a positive control), or E9 at concentrations between 4 μM to 126.4 pM (0.5 log serial dilutions) for 2 hours. THZ531 and E9 are further discussed in Gao et al, Cell Chemical Biology 2017. The amount of phosphorylated RNA polymerase II and the total amount of RNA polymerase II was analyzed using antibodies. The values were normalized by taking the ratio of the phosphorylated RNA polymerase II to the total RNA polymerase II. As depicted in FIG. 3A (THZ531), FIG. 3B (Compound 1), and Table 6, increasing the concentration of compound 1 results in a dose-specific increase in the inhibition of phosphorylation of RNA polymerase II.

TABLE 6
in vitro IC₅₀ values for compound 1 (nM)

| | THZ531* | E9* | Compound 1 |
|---|---|---|---|
| IC50 [nM] | 312.1 | 134.8 | 12.51 |
| Bottom | 9.677 | −7.599 | 0.1958 |
| Top | 83.25 | 88.37 | 85.35 |
| Hill Slope | 1.412 | 1.274 | 1.438 |

### Example 8: Compound 4 and Compound 5 Inhibit Phosphorylation of RNA Polymerase II In Vivo

The ability of compound 4 and compound 5 to inhibit phosphorylation of RNA polymerase II at the S2 unit was analyzed in a mouse xenograft model of triple-negative breast cancer. CB-17 SCID mice were inoculated with the HCC70 cells as the in vivo model for triple negative breast cancer. Each mouse was inoculated subcutaneously on the right flank with HCC70 tumor cells (5×106) in 0.1 mL of RPMI-1640 Medium and Matrigel mixture (1:1 ratio) for tumor development. The treatment started when the mean tumor size reached approximately 100-150 mm³. Mice were treated intravenously with vehicle only, 20 mg/kg of compound 4, 25 mg/kg of compound 4, 10 mg/kg of compound 5, or 25 mg/kg of compound 5 for 2 hours. Measurements were taken at 0.5 hours, 6 hours and 24 hours after the dose. The amount of phosphorylated RNA polymerase II and the total amount of RNA polymerase II was analyzed. The values were normalized by taking the ratio of the phosphorylated RNA polymerase II to the total RNA polymerase II (pS2/S2).

As depicted in FIG. 4, treatment with either dose of compound 4 or compound 5 reduced the pS2/S2 at all time points when compared to treatment with the vehicle alone. Treatment with 25 mg/kg of compound 5 produced a greater decrease in the pS2/S2 ratio than treatment with 10 mg/kg of compound 5 at all time points.

### Example 9: Ex Vivo Analysis of Human and Cynomolgus Peripheral Blood Mononuclear Cells

The ability of the compounds to inhibit phosphorylation of peripheral blood mononuclear cells (PBMCs) was analyzed ex vivo. Compounds 1 and 4 were dosed at concentrations between 4 μM to 126.4 pM (0.5 log serial dilutions) for 2 hours. The amount of phosphorylated RNA polymerase II and the total amount of RNA polymerase II was analyzed. The values were normalized by taking the ratio of the phosphorylated RNA polymerase II to the total RNA polymerase II (pS2/S2).

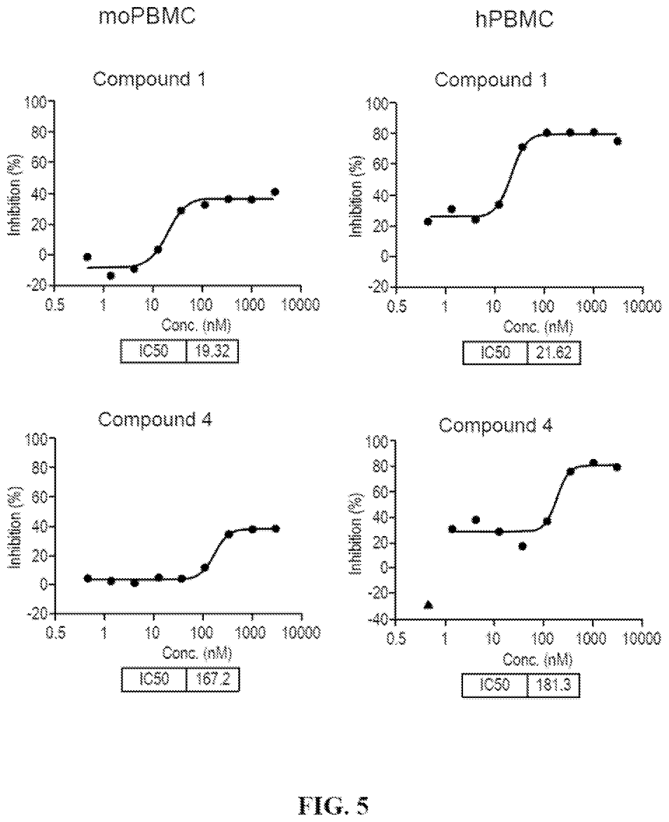

As depicted in FIG. 5, increasing concentrations of compounds resulted in an increase in the percent inhibition. IC₅₀ values were calculated for both human (hPMBC) and cynomolgus (monkey, moPBMC). In hPBMCs, the IC₅₀ values were 19.32 nM for compound 1, 167.2 nM for compound 4. In moPBMCs, the IC₅₀ values were 21.62 nM for compound 1, 181.3 nM for compound 4.

### Example 10: In Vitro Analysis of Gene Expression in Triple-Negative Breast Cancer Cells Treated with Compound 1

HCC70 (ATCC CRL-2315) cells in 96-well plate format were treated with either DMSO or compound 1 for 2 hours. After compound treatment was done for 2 hours, the plates were washed by PBS once, then fresh culture medium was added to the cells and cells were incubated for 4, 10, 22, 46, 70 hours respectively. At each time point (4, 10, 22, 46, 70 hours after 2 h washout) RNA extraction was done according to manufacturer's instructions as follows. RNA Extraction and qPCR was done using the Ambion Cell to CT kit (Cat #AM1728). Briefly, cells were removed from the incubator, washed once in PBS, lysed by adding 50 μL Lysis Solution from the kit, mixed 5 times and 5 μL Stop Solution was added to the well. All actions were done using RNAase and DNAase free labware.

The Reverse Transcription Reaction mix was then prepared. All reagents were kept in an ice-water bath during the whole operation.

| Reagent | Volume (μl) |
|---|---|
| 2 × RT Buffer | 50 |
| 20 × RT Enzyme Mix | 5 |
| RNA | 30 |
| H2O | 15 |
| Total volume | 100 |

A standard Applied Biosystems MiniAmp PCR machine was used with the following procedure to create cDNA template from the cell-based sample:

| | |
|---|---|
| 1. | Heat Lid to 110° C. |

| | | | |
|---|---|---|---|
| 2. | 37° C. | 1 | hour |
| 3. | 95° C. | 5 | min |

| | | |
|---|---|---|
| 4. | 4° C. | infinity |

PCR plates were centrifuged in a precooled plate centrifuge and integrity of the plate cover was observed. The reverse transcription products were stored at −20° C. before analysis by qPCR. Multiplex qPCR was done by Using TaqMan® Gene Expression Master Mix. The reaction mix was prepared as noted below. Three replicates were performed each time for each sample. All reagents were kept in an ice-water bath during the whole operation.

| | | |
|---|---|---|
| 2 × TaqMan ® Universal PCR Master Mix | 5 | μL |
| 20 × target special gene TaqMan probe/primer | 0.5 | μL |
| 20 × target GAPDH or ACTB TaqMan probe/primer | 0.5 | μL |
| cDNA template (from previous step) | 2 | μL |
| H₂O | 2 | μL |
| Total volume | 10 | μL |

The setting of QuantStudio®5 Real-Time qPCR System was shown in the following figure and the fluorescence gain was set at 1×.

| | | | |
|---|---|---|---|
| 1. | Heat Lid | 110° | C. |
| 2. | 50° C. | 2 | min |
| 3. | 95° C. | 10 | min |
| 4. | 40 cycles | | |
| | 95° C. | 15 | sec |
| | 60° C. | 60 | sec |

| | |
|---|---|
| 4° C. | infinity |

Data Analysis was done according to the QuantStudio5 Software. Threshold of signal was calculated by QuantStudio 5 software using the default setting. The relative gene expression was evaluated using the following formulas:

ΔCt=Mean of Ct(target gene)−Mean of Ct(Housekeeping gene)

mRNA level=2−ΔCt

% Vehicle=100×[mRNA(compound treated)/mRNA(vehicle)]

Gene expression was detected using qPCR with the Taqman (Applied Biosystems, ThermoFisher Scientific; Carlsbad, Calif.) probe listed in Table 7. Gene expression was normalized using the ddCt method with a ratio of target gene expression to “house-keeping” control gene(s) ACTB or GAPDH.

TABLE 7
DDR gene set 1

| Gene | | Gene | | Gene | |
|---|---|---|---|---|---|
| Name | TaqMan Probe | Name | TaqMan Probe | Name | TaqMan Probe |
| BRCA1 | Hs02387158_m1 | MCL1 | Hs01050896_m1 | CHEK2 | Hs00200485_m1 |
| BRCA2 | Hs00609073_m1 | MYC | Hs00153408_m1 | ACTB | Hs01060665_g1 |
| ATM | Hs00175892_m1 | B2M | Hs00187842_m1 | GAPDH | Hs02758991_g1 |
| ATR | Hs00992123_m1 | TBP | Hs00427620_m1 | PARP2 | Hs00193931_m1 |
| H2AX | Hs01573336_s1 | GUSB | Hs00939627_m1 | CHEK1 | Hs00967506_m1 |
| RAD51 | Hs00947967_m1 | PARP1 | Hs00242302_m1 | BCL2 | Hs01048932_g1 |
| BCLXL | Hs00236329_m1 | | | | |

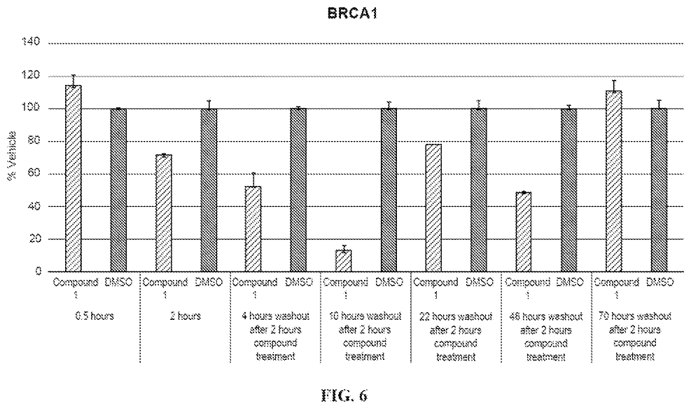

An example of normalized gene expression for one of the DDR genes is shown in FIG. 6. Compound 1 shows a reduction in levels of BRCA1 expression starting at 2 hours after treatment. The reduction in gene expression continues after the washout, with the strongest reduction in gene expression of cells treated with compound 1 seen at 10 hours after treatment (post-washout).

### Example 11: In Vivo Analysis of DDR Gene Expression of Mice Treated with Compound 4

A triple-negative xenograft model of breast cancer was used in this experiment. CB-17 SCID mice were inoculated with the HCC70 cells as the in vivo model for triple negative breast cancer. Each mouse was inoculated subcutaneously on the right flank with HCC70 tumor cells (5×10⁶) in 0.1 mL of RPMI-1640 Medium and Matrigel mixture (1:1 ratio) for tumor development. The treatment started when the mean tumor size reached approximately 100-150 mm³. Mice were treated with a single dose of either vehicle, 20 mg/kg compound 4, or 25 mg/kg of compound 4 intravenously. 0.5, 6, and 24 hours post dose, tumor cells were collected and analyzed for gene expression. The genes tested are listed in Table 8.

TABLE 8
DDR genes analyzed

| Gene Name | TaqMan Probe | Gene Name | TaqMan Probe |
|---|---|---|---|
| BRCA1 | Hs02387158_m1 | MCL1 | Hs01050896_m1 |
| BRCA2 | Hs00609073_m1 | MYC | Hs00153408_m1 |
| ATM | Hs00175892_m1 | PARP1 | Hs00242302_m1 |
| ATR | Hs00992123_m1 | PARP2 | Hs00193931_m1 |
| H2AX | Hs01573336_s1 | CHEK1 | Hs00967506_m1 |
| RAD51 | Hs00947967_m1 | ACTB | Hs01060665_g1 |
| BCLXL | Hs00236329_m1 | GAPDH | Hs02758991_g1 |
| BCL2 | Hs01048932_g1 | | |

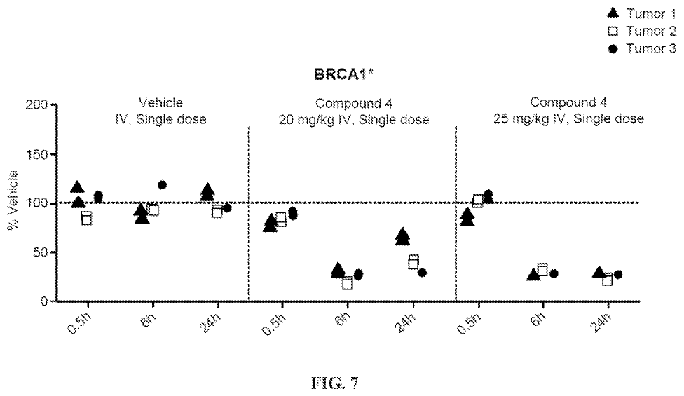

In FIG. 7, gene expression data is shown for BRCA1, one of the genes tested. At 6 and 24 hours after dose administration, BRCA1 expression is reduced in the tumor cells of mice treated with either dose of compound 4.

### Example 12: Clinical Trial Design for Metastatic Castration-Resistant Prostate Cancer (mCRPC)

### Study Description

Brief Summary: The purpose of this study is to find out if a new drug, compound 4, is safe and has beneficial effects when given alone, or in combination with the PARP inhibitor, olaparib, in men with metastatic castration-resistant prostate cancer (mCRPC).

| Condition or disease | Intervention or treatment |
|---|---|
| mCRPC | Compound 4 alone |
| mCRPC | Compound 4 + olaparib |

### Detailed Description:

This phase Ib/II clinical trial will assess the safety, tolerability, RP2D, and preliminary anti-tumor activity of the CDK12/13 inhibitor compound 4, alone, or in combination with olaparib.

The primary objective of the phase Ib study is to establish safety, tolerability, and RP2D. Adverse events (AEs) will be graded according to the National Cancer Institute Common Terminology Criteria for Adverse Events (CTCAE), version 5.0. During the phase Ib portion of the study, a dose limiting toxicity (DLT) will be defined as an AE occurring during Cycle 1 that is attributable to compound 4 and/or olaparib, is unrelated to mCRPC, intercurrent illness, or concomitant medications, and meets at least one criterion from a comprehensive list of DLT criteria based on CTACE, version 5.0. Dose-escalation will continue until DLTs are observed in at least 2 of the patients treated at a dose level, leading to the conclusion that the MTD has been exceeded.

The primary objective of the phase II portion of the study is to estimate the objective response rate (ORR) of compound 4 alone or in combination with olaparib based on RECIST v1.1 or PSA decline ≥50%, as described per Prostate Cancer Working Group 3 (PCWG3). Secondary endpoints in phase II include progression-free survival, disease control rate, duration of response, and time to progression. Additional exploratory endpoints to include evaluation of genetic biomarkers of response and progression.

### Study Design

### Study Type: Interventional Clinical Trial

Estimated Enrollment: 100 patients

### Intervention Model: Single Group Assignment

Masking: None (open label)

### Primary Purpose: Treatment

### Arms and Interventions

| Arm | Intervention |
|---|---|
| Experimental: low dose Compound 4 | Compound 4 |
| Experimental: high dose Compound 4 | Compound 4 |
| Experimental: low dose Compound 4 and | Compound 4 + olaparib |
| olaparib | |

### Outcome Measures

### Primary Outcome Measure:

1. Percentage of patients with treatment emergent adverse events as defined by CTCAE v.4.03 [e.g. Time Frame: Baseline through 1 year]. Number and percentage of patients with treatment emergent adverse events and toxicity based upon CTCAE v.4.03 scoring.
2. Maximum tolerated dose (MTD) of Compound 4 alone and in combination with olaparib as defined by CTCAE 4.03 [Time Frame: Baseline through 1 year]. To determine the MTD of Compound 4 alone and in combination with olaparib during the dose escalation as defined by CTCAE v.4.03.
3. Objective response rate (ORR) of compound 4 alone and in combination with olaparib based on RECIST v1.1 or PSA decline ≥50%, as described per Prostate Cancer Working Group 3 (PCWG3).

### Secondary Outcome Measure:

1. Radiographic progression free survival [Time Frame: Radiographic progression free survival will be evaluated 6 months post trial entry] rPFS will be defined by either RECIST v.1.1 progression and/or progression on bone scan or as described per Prostate Cancer Working Group 3 (PCWG3). It will be measured from the date of trial entry to the first occurrence of radiographic progression or death from any cause
2. Progression free survival [Time Frame: Progression free survival will be evaluated 6 months post trial entry] PFS will be measured from date of trial entry until radiographic progression defined by RECIST v.1.1, as described per Prostate Cancer Working Group 3 (PCWG3) or unequivocal clinical progression or death
3. Time to PSA Progression [Time Frame: Time to PSA progression will be evaluated 6 months post trial entry] For patients who have achieved ≥50% decrease from the cycle 1 day 1 (baseline), the PSA progression date is defined as the date that a ≥25% increase and an absolute increase of ≥2 ng·mL above the nadir is documented. This must be confirmed by a second consecutive value. For patients without a PSA decrease of this magnitude or no decrease at all, PSA progression date is defined as the date that a ≥25% increase and an absolute increase of ≥2 ng/mL above the baseline is documented. This must also be confirmed by a second consecutive value.
4. Duration of PSA response [Time Frame: Duration of PSA response will be evaluated 6 months post trial entry] Duration of PSA response is calculated from the time the PSA value first declines by at least 50% of the cycle 1 day 1 (baseline) value (must be confirmed by a second value) until the time there is an increase of 25% of PSA nadir, provided the absolute increase is at least 2 ng/mL. The increase must be confirmed by a second consecutive measurement.
5. Time to radiographic progression [Time Frame: Will be evaluated 6 months post trial entry] Time to radiographic progression (progression defined by either RECIST V.1.1. progression and/or progression on bone scan or as described per Prostate Cancer Working Group 3 (PCWG3)) will be measured from the date of trial entry to the first occurrence of radiographic progression. Death from prostate cancer or any other cause without prior radiographic evidence of progression will not count as an event.
6. Overall survival [Time Frame: Will be evaluated 6 months post trial entry] OS will be measured from the date of trial entry to the date of death (whatever the cause). Survival time of living patients will be censored on the last date a patient is known to be alive or lost to follow-up
7. PSA objective response [Time Frame: Will be evaluated 6 months post trial entry] PSA response and PSA progression are defined according to the consensus guidelines of the Prostate Cancer Working Group 3 (PCWG3).

### Exploratory Outcome Measure:

To determine clinical correlative biomarkers of response and resistance to compound 4 alone or in combination with olaparib. To this end, circulating tumor cells (CTCs), circulating tumor-associated nucleic acids (i.e. ctDNA) and/or paired normal/tumor tissue specimens will be used to conduct exploratory analysis to identify biologic, genetic and transcriptomic profiles that correlate with response and resistance to compound 4 alone or in combination with olaparib.

### Eligibility Criteria

| | |
|---|---|
| Ages Eligible for Study: | 18 Years and older (Adult, Older Adult) |
| Sexes Eligible for Study: | Male |
| Accepts Healthy Volunteers: | No |

### Criteria

### Inclusion Criteria:

Histologically confirmed prostate adenocarcinoma
Metastatic disease confirmed by bone scan or CT scan
History of bilateral orchiectomies or ongoing GnRH agonist or antagonist
Progressive mCRPC as defined by serum testosterone levels ≤50 ng/dL and disease progression in bone, nodal disease, visceral disease, and/or PSA progression per Prostate Cancer Working Group 3
Prior therapy with either abiraterone or enzalutamide
Prior therapy with docetaxel

### Example 13: Clinical Trial Design for Platinum Resistant, High-Grade Serous Ovarian Cancer Study Description

Brief Summary: The purpose of this study is to find out if a new drug, compound 4, is safe and has beneficial effects when given alone, or in combination with the PARP inhibitor, olaparib, in women with platinum resistant, high-grade serous ovarian cancer (HGSOC).

| Condition or disease | Intervention or treatment |
|---|---|
| Platinum Resistant HGSOC | Compound 4 alone |
| Platinum Resistant HGSOC | Compound 4 + olaparib |

Detailed Description: The primary and secondary objective are to assess the safety of Compound 4 alone, or the combination of Compound 4 and olaparib, in a phase 1/1b trial of patients with platinum-resistant, high-grade serous ovarian cancer; to determine the response rate and percentage of participants who remain progression free survival (PFS) at 6 months (% PFS) among ovarian cancer participants; and to identify potential biological predictors of response and progression of disease with compound 4 alone, or the combination of compound 4 and olaparib.

### Study Design

Study Type: Interventional Clinical Trial

Estimated Enrollment: 60 patients

### Intervention Model: Single Group Assignment

Masking: None (open label)

### Primary Purpose: Treatment

### Arms and Interventions

| Arm | Intervention |
|---|---|
| Experimental: low dose compound 4 | compound 4 |
| Experimental: high dose compound 4 | compound 4 |
| Experimental: low dose compound 4 and | compound 4 and olaparit |
| olaparib | |

### Outcome Measures

### Primary Outcome Measure:

1. Percentage of patients with treatment emergent adverse events as defined by CTCAE v.4.03 [Time Frame: Baseline through 1 year]. Number and percentage of patients with treatment emergent adverse events and toxicity based upon CTCAE v.4.03 scoring.
2. Maximum tolerated dose (MTD) of compound 4 alone and in combination with olaparib as defined by CTCAE 4.03 [Time Frame: Baseline through 1 year]. To determine the MTD of compound 4 and olaparib during the dose escalation as defined by CTCAE v.4.03.
2. Percentage progression free survival (PFS) as defined by RECIST v.1.1 [Time Frame: Baseline through 6 months]. To determine percentage of patients who remain progression free at 6 months (% PFS) among platinum resistant HGSOC patients (defined as progression within 6 months after last platinum regimen) who are treated with compound 4 alone, or compound 4+olaparib as defined by RECIST v.1.1.

### Secondary Outcome Measure:

1. Overall Response Rate (ORR) as defined by RECIST v.1.1 [Time Frame: Baseline through 6 months]. To determine response rate among platinum resistant (defined as progression within 6 months after last platinum regimen) HGSOC cancer who are treated with compound 4 alone, or compound 4+olaparib as defined by RECIST v.1.1.
2. Overall Survival (OS) as defined by RECIST v.1.1 [Time Frame: Baseline through 6 months]. To determine response rate among platinum resistant (defined as progression within 6 months after last platinum regimen) HGSOC patients who are treated with compound 4 alone, or compound 4+olaparib as defined by RECIST v.1.1.

### Exploratory Outcome Measure:

To determine clinical correlative biomarkers of response and resistance to compound 4 alone or in combination with olaparib. To this end, circulating tumor cells (CTCs), circulating tumor-associated nucleic acids (i.e. ctDNA) and/or paired normal/tumor tissue specimens will be used to conduct exploratory analysis to identify biologic, genetic and transcriptomic profiles that correlate with response and resistance to compound 4 alone or in combination with olaparib.

### Eligibility Criteria

| | |
|---|---|
| Ages Eligible for Study: | 18 Years and older (Adult, Older Adult) |
| Sexes Eligible for Study: | Female |
| Accepts Healthy Volunteers: | No |

### Criteria

### Inclusion Criteria:

Patients must have been diagnosed with advanced and histologically confirmed high-grade serous ovarian, primary peritoneal or fallopian tube cancer
Patients must have platinum resistant ovarian cancer, defined as progression within 6 months after last platinum regimen.
Patients must have at least one lesion that meets the definition of measurable disease by RECIST v1.1.
Female patients ≥18 years of age
Eastern Cooperative Oncology Group (ECOG) performance status (PS) 0
Documented germline and somatic BRCA1/2 and DNA damage gene (DDR) mutation status
Availability of sufficient archival and/or freshly biopsied normal/tumor specimens for confirmatory and exploratory biomarker analysis

### Exclusion Criteria:

Is currently participating and receiving study therapy or has participated in a study of an investigational agent and received study therapy or used an investigational device within 4 weeks of the first dose of treatment.
Patients cannot have had primary platinum refractory cancer, i.e. documented cancer progression while receiving platinum or within one month of receipt of a platinum-based regimen.
No line limit but patients must have received no more than 1 prior regimens in the platinum resistant setting.
Has had prior chemotherapy, targeted small molecule therapy, or radiation therapy within 2 weeks prior to study Day 1 or who has not recovered (i.e., ≤Grade 1 or at baseline) from adverse events due to a previously administered agent.
No prior targeted DNA damage repair (DDR) inhibitors and no prior gemcitabine as a single agent; hormonal therapies and antiangiogenic therapies (as single agents) and PARP-inhibitors as maintenance therapy do not count as separate lines.
Has a known additional malignancy that is progressing or requires active treatment. In addition, patients cannot have been diagnosed with another malignancy within 3 years of starting treatment. Exceptions include fully resected basal cell carcinoma of the skin or squamous cell carcinoma of the skin, in situ cervical cancer, fully resected ductal carcinoma in situ, and stage IA, noninvasive grade I endometrioid endometrial cancer, that has undergone curative therapy.
Has known active central nervous system (CNS) metastases and/or carcinomatous meningitis. Participants with previously treated brain metastases may participate provided they are stable (without evidence of progression by imaging for at least four weeks prior to the first dose of trial treatment and any neurologic symptoms have returned to baseline), have no evidence of new or enlarging brain metastases, and are not using steroids for at least 7 days prior to trial treatment. This exception does not include clinically active and significant carcinomatous meningitis that is excluded regardless of clinical stability.
Has an active infection requiring systemic therapy
Has a history or current evidence of any condition, therapy, or laboratory abnormality that might confound the results of the trial, interfere with the participant's participation for the full duration of the trial, or is not in the best interest of the participant to participate, in the opinion of the treating investigator.

### Example 14: Clinical Trial Design for Triple Negative Breast Cancer

### Study Description

Brief Summary: The purpose of this study is to find out if a new investigational drug, Compound 4, is safe and has beneficial effects when given alone, or in combination with the PARP inhibitor, olaparib, in patients with metastatic triple (ER−, PR− and HER2−) negative breast cancer (TNBC) as defined by the eligibility criteria.

| Condition or disease | Intervention or treatment |
|---|---|
| TNBC | Compound 4 alone |
| TNBC | Compound 4 + olaparib |

### Detailed Description:

This phase Ib/II clinical trial will assess the safety, tolerability, RP2D, and preliminary anti-tumor activity of the new investigational drug, compound 4, either alone, or in combination with the PARP inhibitor, olaparib, in the treatment of patients with metastatic triple negative breast cancer (TNBC).

The primary objective of the phase Ib study is to establish safety, tolerability, and RP2D. Adverse events (AEs) will be graded according to the National Cancer Institute Common Terminology Criteria for Adverse Events (CTCAE), version 5.0. During the phase Ib portion of the study, a dose limiting toxicity (DLT) will be defined as an AE occurring during Cycle 1 that is attributable to compound 4 and/or olaparib, is unrelated to TNBC, intercurrent illness, or concomitant medications, and meets at least one criterion from a comprehensive list of DLT criteria based on CTACE, version 5.0. Dose-escalation will continue until DLTs are observed in at least 2 of the patients treated at a dose level, leading to the conclusion that the MTD has been exceeded.

The primary objective of the phase II portion of the study is to estimate the objective response rate (ORR) of compound 4, either alone or, in combination with olaparib, based on RECIST v1.1 criteria. Secondary endpoints in phase II include progression-free survival, clinical benefit rate/disease control rate, duration of response, and time to progression. Additional exploratory endpoints to include evaluation of genetic biomarkers of response and progression.

### Study Design

### Study Type: Interventional Clinical Trial

Estimated Enrollment: 100 patients

### Intervention Model: Single Group Assignment

Masking: None (open label)

### Primary Purpose: Treatment

### Arms and Interventions

| Arm | Intervention |
|---|---|
| Experimental: low dose Compound 4 | Compound 4 |
| Experimental: high dose Compound 4 | Compound 4 |
| Experimental: low dose Compound 4 and | Compound 4 and olaparib |
| olaparib | |

### Outcome Measures

### Primary Outcome Measure:

1. Percentage of patients with treatment emergent adverse events as defined by CTCAE v.4.03 [e.g. Time Frame: Baseline through 1 year]. Number and percentage of patients with treatment emergent adverse events and toxicity based upon CTCAE v.4.03 scoring; [Time Frame: Up to 3 months post treatment] Incidence will be determined for participants with TNBC that received at least one dose of Compound 4 and/or olaparib.
2. Maximum tolerated dose (MTD) of Compound 4 alone and in combination with olaparib as defined by CTCAE 4.03 [Time Frame: Baseline through 1 year]. To determine the MTD of Compound 4 and olaparib during the dose escalation as defined by CTCAE v.4.03.
3. Objective response rate (ORR) of compound 4 alone or in combination with olaparib will be determined based on RECIST v.1.1. criteria.

### Secondary Outcome Measure:

1. Clinical benefit rate (CBR) or disease control rate (DCR) for Compound 4 alone or in combination with olaparib [Time Frame: Up to 6 months post treatment] will be determined based on RECIST v.1.1. criteria. Participants who achieve a complete response (CR), partial response (PR), or stable disease (SD) for at least 6 months on the current protocol will be qualified as deriving benefit from therapy, and will count towards the CBR/DCR measurement.
2. Progression-free survival (PFS) for Compound 4 alone or in combination with olaparib [Time Frame: Up to 1 year post treatment] PFS is defined as the time from first treatment with olaparib (i.e., cycle 1 day 1) to the first of either recurrence or relapse (anywhere in the body), based on RECIST v.1.1. criteria or death at time of last follow-up at 12-months.
3. Overall survival (OS) Compound 4 alone or in combination with olaparib [Time Frame: Up to 1 year post treatment] Based on RECIST v.1.1. criteria, OS will be defined as the time from first treatment with Compound 4 alone or in combination with olaparib (i.e., Day 1) to the date of death or last follow-up at 12 months.
4. Duration of response (DOR) for Compound 4 alone or in combination with olaparib [Time Frame: Up to 6 months post treatment] based on RECIST v.1.1. criteria. The duration of overall response is measured from the time measurement criteria are met for CR or PR (whichever is first recorded) until the first date that recurrent or progressive disease is objectively documented taking as reference for progressive disease the smallest measurements recorded since the treatment started. The duration of overall CR is measured from the time measurement criteria are first met for CR until the first date that progressive disease is objectively documented. If a participant dies, irrespective of cause, without documentation of recurrent or progressive disease beforehand, then the date of death will be used to denote the response end date.

### Exploratory Outcome Measure:

To determine clinical correlative biomarkers of response and resistance to Compound 4 alone or in combination with olaparib. To this end, circulating tumor cells (CTCs), circulating tumor-associated nucleic acids (i.e. ctDNA) and/or paired normal/tumor tissue specimens will be used to conduct exploratory analysis to identify biologic, genetic and transcriptomic profiles that correlate with response and resistance to Compound 4 alone or in combination with olaparib.

### Eligibility Criteria

| | |
|---|---|
| Ages Eligible for Study: | 18 Years and older (Adult, Older Adult) |
| Sexes Eligible for Study: | Male and Female |
| Accepts Healthy Volunteers: | No |

### Criteria

### Inclusion Criteria:

Ability to understand and the willingness to sign a written informed consent document.
Participants are >=18 years old at time of informed consent.
Metastatic TNBC, as defined by:
   1. ER and PR negative as defined as ER<10% and PR<10% by immunohistochemistry according to American Society of Clinical Oncology (ASCO)/College of American Pathologists (CAP) guidelines for hormone receptor testing
   2. HER2 non-amplified per ASCO/CAP guidelines, defined as:
      a. IHC score 0/1+
      b. IHC 2+ and in situ hybridization (ISH) non-amplified with a ratio of HER2 to CEP17<2.0, and if reported, average HER2 gene copy number <4 signals/cells; or
      c. ISH non-amplified with a ratio of HER2 to CEP17<2.0, and if reported, average HER2 gene copy number <4 signals/cells
Participants must have at least one measurable site of disease as defined by RECIST v1.1 that is amendable to biopsy.
Prior therapies for metastatic breast cancer
   1. Frontline patients who have not received prior systemic therapy for metastatic breast cancer are eligible.
   2. Patients who have received <=2 prior chemotherapy regimens for metastatic breast cancer are eligible.
Participants must have fully recovered from the acute toxic effects of all prior treatment to grade 1 or less, except alopecia and <=Grade 2 neuropathy which are allowed
Participants must have a life expectancy >=16 weeks.
Participant must have Eastern Cooperative Oncology Group (ECOG) performance status <=1
Participant must consent to undergo a pre-treatment screening biopsy for enrollment and subsequent biomarker analyses.
Participants must consent to undergo one mandatory on-study tumor biopsy following a 4 week, single cycle induction treatment of olaparib. A second on-study biopsy at time of disease progression is optional, but not mandatory.
Participants must not have had prior immunotherapy with anti-PD-L1, including durvalumab anti-PD-1, anti-CTLA4 or similar drugs.
Participants must have normal organ and bone marrow function measured within 28 days prior to administration of study treatment as defined below:
   1. Haemoglobin >=10.0 g/dL with no blood transfusion in the past 28 days
   2. Absolute neutrophil count (ANC)>=1.5×109/L
   3. Platelet count >=100×109/L
   4. Total bilirubin <=1.5× institutional upper limit of normal (ULN)
   5. Aspartate aminotransferase (AST) (Serum Glutamic Oxaloacetic Transaminase (SGOT))/Alanine aminotransferase (ALT) (Serum Glutamic Pyruvate Transaminase (SGPT))<=2.5× institutional upper limit of normal unless liver metastases are present in which case they must be <=5×ULN
6. Participants must have creatinine clearance estimated of >=51 mL/min using the Cockcroft-Gault equation or based on a 24 hour urine test: Estimated creatinine clearance=(140-age [years])×weight (kg) (×F) serum creatinine (mg/dL)×72; where F=0.85 for females and F=1 for males.
Female participants of childbearing potential must have a negative urine or serum pregnancy test within 72 hours prior to receiving the first dose of study medication. If the urine test is positive or cannot be confirmed as negative, a serum pregnancy test will be required.
Female participants of childbearing potential agree to use adequate methods of contraception starting with the first dose of study therapy through 60 days after the last dose of study therapy. Participants of childbearing potential are those who are not proven postmenopausal. Postmenopausal is defined as:
   1. Amenorrheic for 1 year or more following cessation of exogenous hormonal treatments
   2. Luteinizing hormone (LH) and Follicle stimulating hormone (FSH) levels in the post menopausal range for women under 50
   3. Radiation-induced oophorectomy with last menses >1 year ago
   4. Chemotherapy-induced menopause with >1 year interval since last menses
   5. Surgical sterilisation (bilateral oophorectomy or hysterectomy)
Male participants must agree to use an adequate method of contraception starting with the first dose of study therapy through 60 days after the last dose of study therapy.
Participants must not have received live vaccines within 30 days prior to the first dose of immunotherapy. Examples of live vaccines include, but are not limited to, the following: measles, mumps, rubella, chicken pox, shingles, yellow fever, rabies, BCG, and typhoid (oral) vaccine. Seasonal influenza vaccines for injection are generally killed virus vaccines and are allowed; however, intranasal influenza vaccines (e.g., Flu-Mist®) are live attenuated vaccines, and are not allowed. Patients, if enrolled, should not receive live vaccine whilst receiving immunotherapy and up to 30 days after the last dose of immunotherapy
Availability of sufficient archival and/or freshly biopsied normal/tumor specimens for confirmatory and exploratory biomarker analysis
Availability of DNA damage repair/response gene (DDR) mutational status including, but not limited to ATM, ATR, BRCA1/2, CDK12, etc.

### Exclusion Criteria:

Currently participating and receiving study therapy or has participated in a study of an investigational agent and received study therapy or used an investigation device within 4 weeks of first dose of treatment; Individuals in the follow-up phase of a prior investigational study may participate as long as it has been 4 weeks since last dose of the previous investigational agent of device.
Other malignancy unless curatively treated with no evidence of disease for >=5 years except: adequately treated non-melanoma skin cancer, curatively treated in situ cancer of the cervix, ductal carcinoma in situ (DCIS), Stage 1, grade 1 endometrial carcinoma. Participants with a history of localized triple negative breast cancer may be eligible, provided they completed their adjuvant chemotherapy more than three years prior to registration, and that the participant remains free of recurrent or metastatic disease
Participants with myelodysplastic syndrome/acute myeloid leukemia or with features suggestive of MDS/AML.
Participant received prior chemotherapy or any other targeted therapies within the past 28, or radiation (except for palliative reasons) within the past 3 weeks, prior to going on-study.
Participants with known active central nervous system (CNS) metastases and/or carcinomatous meningitis.
Participants with previously treated brain metastases may participate provided they are stable [without evidence of progression by imaging (confirmed by CT scan if CT used at prior imaging, or confirmed by MRI if MRI was used at prior imaging) for at least four weeks prior to the first dose of trial treatment and any neurologic symptoms have returned to baseline], have no evidence of new or enlarging brain metastases, and are not using steroids for at least 7 days prior to trial treatment. This exception does not include carcinomatous meningitis which is excluded regardless of clinical stability.
Participants unable to swallow orally administered medication and participants with gastrointestinal disorders likely to interfere with absorption of the study medication
Participants with visceral crisis defined as severe organ dysfunction as assessed by signs and symptoms, laboratory studies, and rapid progression of disease.
Active infection requiring systemic antibiotic therapy. Participants requiring systemic antibiotics for infection must have completed therapy before treatment is initiated.
Participants considered a poor medical risk due to a serious, uncontrolled medical disorder, non-malignant systemic disease or active, uncontrolled infection. Examples include, but are not limited to, uncontrolled ventricular arrhythmia, recent (within 3 months) myocardial infarction, uncontrolled major seizure disorder, unstable spinal cord compression, superior vena cava syndrome, extensive interstitial bilateral lung disease on High Resolution Computed Tomography (HRCT) scan or any psychiatric illness/social situation that prohibits obtaining informed consent.
Resting ECG indicating uncontrolled, potentially reversible cardiac conditions, as judged by the investigator (e.g., unstable ischemia, uncontrolled symptomatic arrhythmia, congestive heart failure, QTcF prolongation >500 ms, electrolyte disturbances, etc.), or participants with congenital long QT syndrome
Participants with a history of hypersensitivity reactions to study agent or their excipients.
Participant is pregnant or breastfeeding, or expecting to conceive or father children within the projected duration of the trial, starting with the screening visit through 120 days after the last dose of trial treatment.
Involvement in the planning and/or conduct of the study
Judgment by the investigator that the patient should not participate in the study if the patient is unlikely to comply with study procedures, restrictions and requirements.

What is claimed is:

1. A method of treating a triple-negative breast cancer in an individual in need thereof, comprising administering to the individual a compound of Formula (I), or pharmaceutically acceptable salt or solvate thereof, wherein the compound of Formula (I) has the structure: 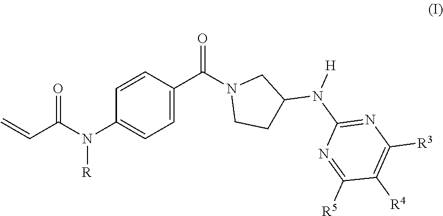
wherein,
R is hydrogen or C1-C3 alkyl;
R³ is selected from hydrogen, halogen, —CN, and optionally substituted C1-C3 alkyl;
R⁴ is selected from selected from halogen, —CN, optionally substituted alkyl, optionally substituted fluoroalkyl, optionally substituted alkenyl, optionally substituted alkynyl; and
R⁵ is hydrogen or optionally substituted alkoxy.

2. The method of claim 1, wherein the triple-negative breast cancer is a metastatic triple-negative breast cancer.

3. The method of claim 1, wherein the triple-negative breast cancer is a non-metastatic triple-negative breast cancer.

4. The method of any one of claims 1-3 wherein the triple-negative breast cancer comprises a basal-like tumor.

5. The method of any one of claims 1-4, wherein the individual has a BRCA1 mutation.

6. The method of any one of claims 1-5, wherein the individual has a BRCA2 mutation.

7. The method of any one of claims 1-6, further comprising:
a) monitoring the phosphorylation state of a CDK12/13 substrate in tumor or normal tissue before and after administration of the compound;
b) determining a ratio of the two values; and
c) observing a decrease in the level of phosphorylated CDK12/13 substrate relative to the total level after administration of the compound.

8. The method of claim 7, wherein the CDK12/13 substrate is RNA polymerase II.

9. The method of any one of claims 1-6, further comprising:
a) monitoring the level of expression of a DNA damage response gene in the tumor;
b) observing a decrease in the level of expression of the DNA damage response gene after administration of the compound; and
c) monitoring the increase in the extent of DNA damage in tumor tissue, cells or circulating tumor cell DNA after administration of the compound.

10. A method of treating ovarian cancer in an individual in need thereof, comprising administering to the individual a compound of Formula (I), or pharmaceutically acceptable salt or solvate thereof, wherein the compound of Formula (I) has the structure: 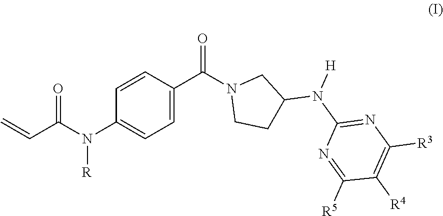
wherein,
R is hydrogen or C1-C3 alkyl;
R³ is selected from hydrogen, halogen, —CN, and optionally substituted C1-C3 alkyl;
R⁴ is selected from selected from halogen, —CN, optionally substituted alkyl, optionally substituted fluoroalkyl, optionally substituted alkenyl, optionally substituted alkynyl; and
R⁵ is hydrogen or optionally substituted alkoxy.

11. The method of claim 10, wherein the cancer comprises a metastatic ovarian cancer.

12. The method of claim 10, wherein the cancer comprises a non-metastatic ovarian cancer.

13. The method of any one of claims 10-12 wherein the ovarian cancer is a high-grade tumor.

14. The method of any one of claims 9-13, wherein the ovarian cancer is recurrent ovarian cancer.

15. The method of any one of claims 10-14, wherein the cancer comprises an ovarian epithelial cancer, a germ cell tumor, a stromal tumor, or an ovarian sarcoma.

16. The method of claim 15, wherein the ovarian sarcoma is an adenosarcoma, leiomyosarcoma or a fibrosarcoma.

17. The method of any one of claims 10-16, wherein the individual has a BRCA1 mutation.

18. The method of any one of claims 10-17, wherein the individual has a BRCA2 mutation.

19. The method of any one of claims 10-18, further comprising:
a) monitoring the phosphorylation state of a CDK12/13 substrate in tumor or normal tissue before and after administration of the compound;
b) determining a ratio of the two values; and
c) observing a decrease in the level of phosphorylated CDK12/13 substrate relative to the total level after administration of the compound.

20. The method of claim 19, wherein the CDK12/13 substrate is RNA polymerase II.

21. The method of any one of claims 10-18, further comprising:
a) monitoring the level of expression of a DNA damage response gene in the tumor;
b) observing a decrease in the level of expression of the DNA damage response gene after administration of the compound; and
c) monitoring the increase in the extent of DNA damage in tumor tissue, cells or circulating tumor cell DNA after administration of the compound.

22. A method of treating castration-resistant prostate cancer in an individual in need thereof, comprising administering to the individual a compound of Formula (I), or pharmaceutically acceptable salt or solvate thereof, wherein the compound of Formula (I) has the structure: 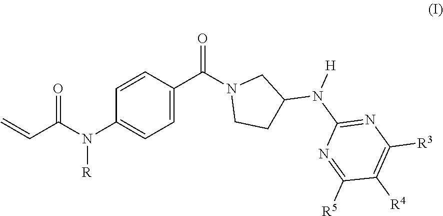
wherein,
R is hydrogen or C1-C3 alkyl;
R³ is selected from hydrogen, halogen, —CN, and optionally substituted C1-C3 alkyl;
R⁴ is selected from selected from halogen, —CN, optionally substituted alkyl, optionally substituted fluoroalkyl, optionally substituted alkenyl, optionally substituted alkynyl; and
R⁵ is hydrogen or optionally substituted alkoxy.

23. The method of claim 22, wherein the castration-resistant prostate cancer comprises a metastatic prostate cancer.

24. The method of claim 22 or 23, wherein the castration-resistant prostate cancer comprises a non-metastatic prostate cancer.

25. The method of any one of claims 22-24, wherein the castration-resistant prostate cancer comprises acinar adenocarcinoma, ductal adenocarcinoma, transitional cell cancer, squamous cell cancer, small cell prostate cancer, a neuroendocrine cancer, or a sarcoma.

26. The method of any one of claims 22-25, wherein the individual has a BRCA1 mutation.

27. The method of any one of claims 22-26, wherein the individual has a BRCA2 mutation.

28. The method of any one of claims 22-27, further comprising:
a) monitoring the phosphorylation state of a CDK12/13 substrate in tumor or normal tissue before and after administration of the compound;
b) determining a ratio of the two values; and
c) observing a decrease in the level of phosphorylated CDK12/13 substrate relative to the total level after administration of the compound.

29. The method of claim 28, wherein the CDK12/13 substrate is RNA polymerase II.

30. The method of any one of claims 22-27, further comprising:
a) monitoring the level of expression of a DNA damage response gene in the tumor;
b) observing a decrease in the level of expression of the DNA damage response gene after administration of the compound; and
c) monitoring the increase in the extent of DNA damage in tumor tissue, cells or circulating tumor cell DNA after administration of the compound.

31. The method of any one of claim 7-9, 19-22, or 28-30, wherein the decrease in the level of phosphorylated RNA polymerase II relative to the total level of RNA polymerase II and/or the decrease in the level of expression of the DNA damage response gene after administration of the compound correlates to an efficacy of the treatment.

32. The method of any one of claim 7-9, 19-22, or 28-31, wherein the monitoring sample is derived from PBMC cells.
